(19) United States
(12) Patent Application Publication
(10) Pub. No.: US 2021/0161511 A1
(43) Pub. Date: Jun. 3, 2021

(54) ULTRASONIC DIAGNOSTIC APPARATUS

(71) Applicant: CANON MEDICAL SYSTEMS CORPORATION, Otawara-shi (JP)

(72) Inventors: Hiroki YOSHIARA, Utsunomiya (JP); Tomohisa IMAMURA, Nasushiobara (JP); Akihiro KAKEE, Nasushiobara (JP); Koichiro KURITA, Nasushiobara (JP)

(73) Assignee: CANON MEDICAL SYSTEMS CORPORATION, Otawara-shi (JP)

(21) Appl. No.: 17/105,772

(22) Filed: Nov. 27, 2020

(30) Foreign Application Priority Data
Nov. 29, 2019 (JP) 2019-217150

(51) Int. Cl. A61B 8/08; A61B 8/00

(52) U.S. Cl. CPC A61B 8/5207; A61B 8/469; A61B 8/4281

(57) ABSTRACT

The ultrasonic diagnostic apparatus according to the present embodiment includes an evaluating circuit, a frequency setting circuit, and a drive circuit. The evaluating circuit is configured to analyze a received signal of a predetermined depth based on received signals of ultrasonic wave to evaluate a degree of beam penetration to a deep portion. The frequency setting circuit is configured to set a transmission frequency based on a result evaluated by the evaluating circuit. The drive circuit is configured to generate a drive pulse based on the set transmission frequency.

### CROSS-REFERENCE TO RELATED APPLICATION

This application is based upon and claims the benefit of priority from Japanese Patent Application No. 2019-217150, filed on Nov. 29, 2019, the entire contents of which are incorporated herein by reference.

### FIELD

An embodiment disclosed in the present specification and drawings relates to an ultrasonic diagnostic apparatus.

### BACKGROUND

In the medical field, an ultrasonic diagnostic apparatus is used for imaging the inside of a subject using ultrasonic waves generated by multiple transducers (piezoelectric vibrators) of an ultrasonic probe. The ultrasonic diagnostic apparatus causes the ultrasonic probe, which is connected to the ultrasonic diagnostic apparatus, to transmit ultrasonic waves into the subject, generates a reception signal based on a reflected wave, and acquires a desired ultrasonic image by image processing.

There are some methods of generating an ultrasonic image in the ultrasonic diagnostic apparatus. In the first method, a radio frequency (RF) signal, which is a reception signal, is delayed and added, a quadrature detection (demodulation) is performed, and a conversion to an I/Q signal composed of an “I (In-phase)” signal and a “Q (Quadrature-phase)” signal is performed. In the second method, a quadrature detection of an RF signal is performed, an I/Q signal is converted to baseband, and a delay addition is performed. The former method is also called “RF beamforming”. The latter method is also called “I/Q beamforming”. Functions for improving the image quality of an ultrasonic image in the I/Q beamforming include a function of controlling a gain of an amplifier, a function of controlling a reception delay curve of a delay control circuit, etc.

### BRIEF DESCRIPTION OF THE DRAWINGS

FIG. 1 is a schematic diagram showing a configuration of an ultrasonic diagnostic apparatus according to a first embodiment.

Each of FIGS. 2A and 2B is a conceptual diagram for explaining a target frequency characteristic having the substantially flat bandwidth close to a designed frequency characteristic in the ultrasonic diagnostic apparatus according to the first embodiment.

Each of FIGS. 3A to 3E is a conceptual diagram for explaining a method of setting a reception filter in the ultrasonic diagnostic apparatus according to the first embodiment.

FIG. 4 is a block diagram showing a configuration of a receiving circuit provided in a transmitting/receiving circuit in the ultrasonic diagnostic apparatus according to the first embodiment.

FIG. 5 is a diagram showing an operation of the ultrasonic diagnostic apparatus according to the first embodiment as a flowchart.

FIG. 6 is a diagram showing an operation of the ultrasonic diagnostic apparatus according to the first embodiment as a flowchart.

Each of FIGS. 7A and 7B is a diagram showing a complex reception filter of the region of interest in the ultrasonic diagnostic apparatus according to the first embodiment.

Each of FIGS. 8A and 8B is a diagram showing a complex reception filter of each region of interest in the ultrasonic diagnostic apparatus according to the first embodiment.

FIG. 9 is a diagram showing, as a frequency characteristic, an effect acquired when a complex reception filter is applied to an I/Q signal in a predetermined region of interest in the ultrasonic diagnostic apparatus according to the first embodiment.

Each of FIGS. 10A and 10B is a diagram showing an effect acquired when a complex reception filter is applied to an I/Q signal in a predetermined region of interest as an ultrasonic image in the ultrasonic diagnostic apparatus according to the first embodiment.

FIG. 11 is a diagram for explaining a frequency compound in the ultrasonic diagnostic apparatus according to the first embodiment.

FIG. 12 is a diagram showing a method of selecting a predetermined region of interest from set multiple regions of interest of the same depth in the ultrasonic diagnostic apparatus according to the first embodiment.

FIG. 13 is a schematic view showing a configuration of the ultrasonic diagnostic apparatus according to a second embodiment.

FIG. 14 is a block diagram showing a configuration of a transmitting/receiving circuit of the ultrasonic diagnostic apparatus according to the second embodiment.

FIG. 15 is a diagram showing an operation of the ultrasonic diagnostic apparatus according to the second embodiment as a flowchart.

FIG. 16 is a diagram showing an operation of the ultrasonic diagnostic apparatus according to the second embodiment as a flowchart.

FIG. 17 is a diagram showing an example of a shallow determination region set in the image region formed by B-mode data for one frame in the ultrasonic diagnostic apparatus according to the second embodiment.

FIG. 18 is a diagram for explaining a method of determining a structure and a parenchyma based on B-mode data for one frame in the ultrasonic diagnostic apparatus according to the second embodiment.

FIG. 19 is a diagram showing an example of a deep determination region set in the image region formed by B-mode data for one frame in the ultrasonic diagnostic apparatus according to the second embodiment.

Each of FIGS. 20A and 20B is a diagram showing an ultrasonic image when the transmission frequency is controlled.

FIG. 21 is a schematic diagram showing a configuration of an ultrasonic diagnostic apparatus according to a third embodiment.

FIG. 22 is a block diagram showing a configuration of a transmitting/receiving circuit in the ultrasonic diagnostic apparatus according to the third embodiment.

FIG. 23 is a diagram showing an operation of the ultrasonic diagnostic apparatus according to the third embodiment as a flowchart.

FIG. 24 is a diagram showing an operation of the ultrasonic diagnostic apparatus according to the third embodiment as a flowchart.

Each of FIGS. 25A and 25B is a diagram showing an ultrasonic image when the transmission frequency and the complex reception filter are controlled.

### DETAILED DESCRIPTION

An ultrasonic diagnostic apparatus according to a present embodiment will be described with reference to the accompanying drawings.

The ultrasonic diagnostic apparatus according to the present embodiment includes an evaluating circuit, a frequency setting circuit, and a drive circuit. The evaluating circuit is configured to analyze a received signal of a predetermined depth based on received signals of ultrasonic wave to evaluate a degree of beam penetration to a deep portion. The frequency setting circuit is configured to set a transmission frequency based on a result evaluated by the evaluating circuit. The drive circuit is configured to generate a drive pulse based on the set transmission frequency.

1. Ultrasonic Diagnostic Apparatus according to First embodiment.

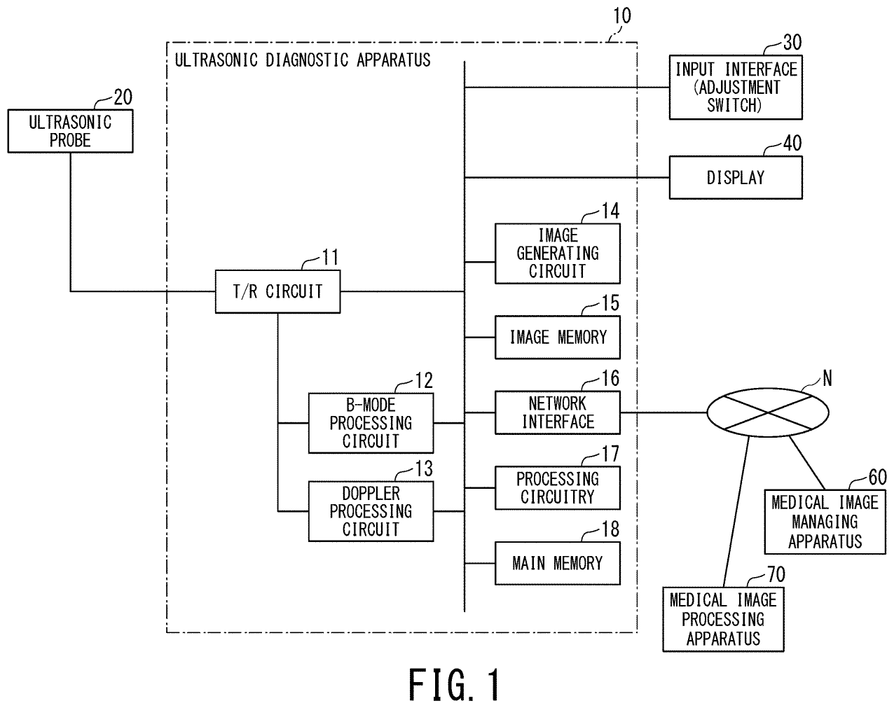

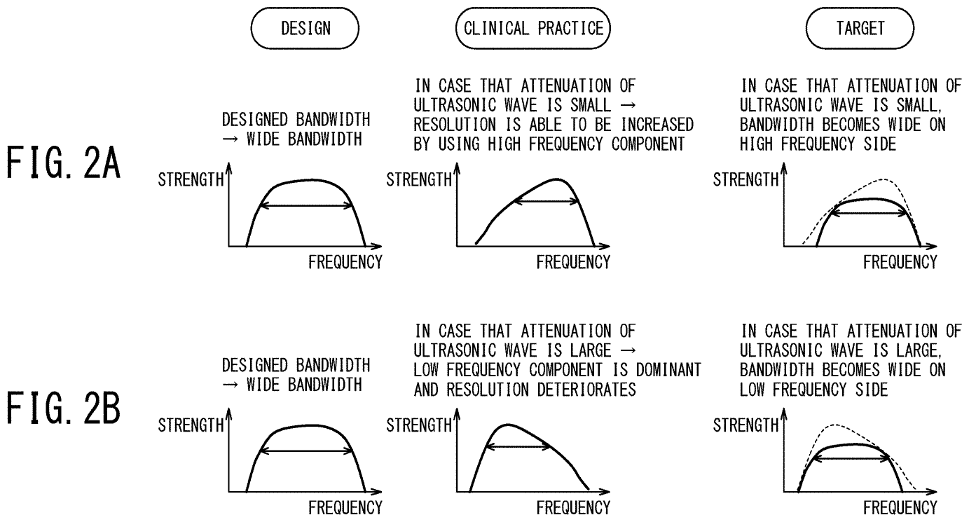

FIG. 1 is a schematic diagram showing a configuration of an ultrasonic diagnostic apparatus according to a first embodiment.

FIG. 1 shows an ultrasonic diagnostic apparatus 10 according to a first embodiment. FIG. 1 shows an ultrasonic probe 20, an input interface 30, and a display 40. Note that an apparatus in which at least one of the ultrasonic probe 20, the input interface 30 and the display 40 are added to the ultrasonic diagnostic apparatus 10 may be referred to as “ultrasonic diagnostic apparatus”. In the following description, a case will be described in which the ultrasonic probe 20, the input interface 30 and the display 40 are all provided outside the ultrasonic diagnostic apparatus 10.

The ultrasonic diagnostic apparatus 10 includes a transmitting/receiving (T/R) circuit 11, a B-mode processing circuit 12, a Doppler processing circuit 13, an image generating circuit 14, an image memory 15, a network interface 16, processing circuitry 17, and a main memory 18. The circuits 11 to 14 are configured by application-specific integrated circuits (ASICs) and the like. However, the present invention is not limited to this case, and all or part of the functions of the circuits 11 to 14 may be realized by the processing circuitry 17 executing a program.

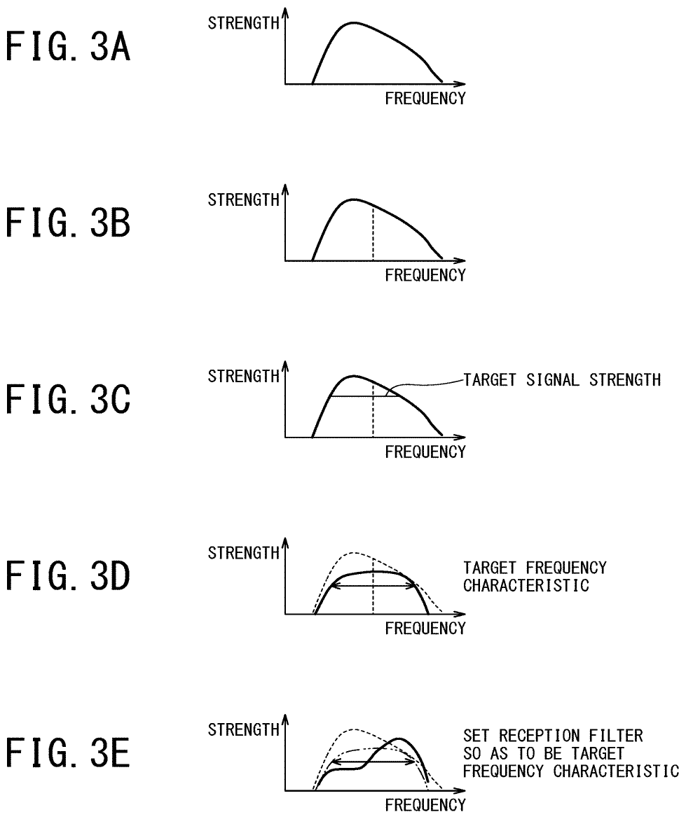

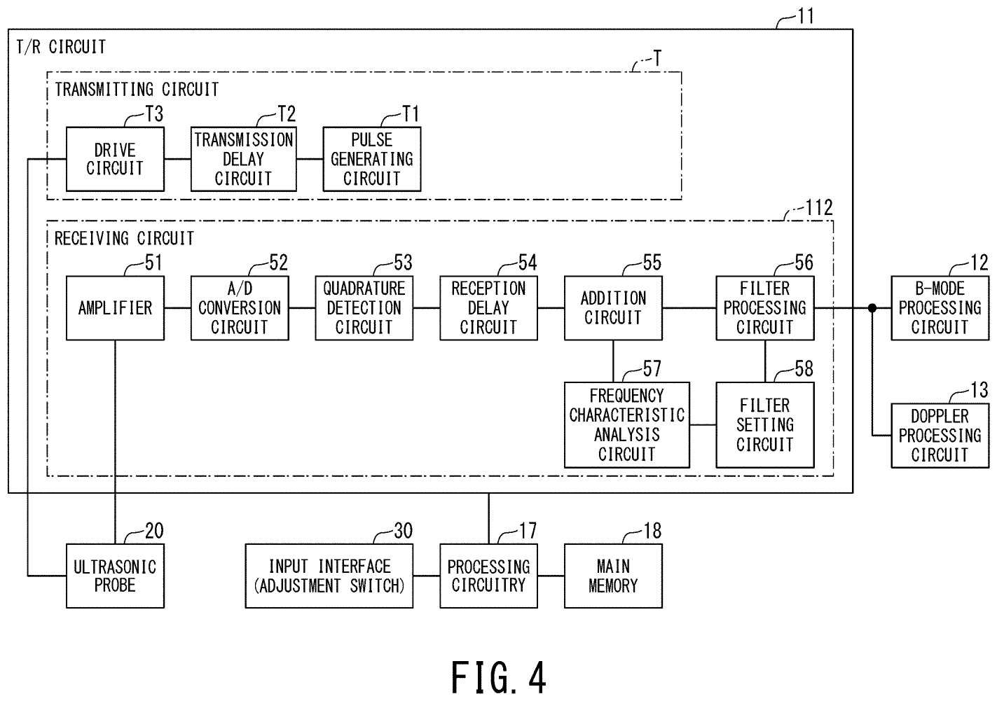

The T/R circuit 11 has a transmitting circuit T and a receiving circuit 112 (shown in FIG. 4). Under the control of the processing circuitry 17, the T/R circuit 11 controls transmission directivity and reception directivity in transmission and reception of ultrasonic waves. The case where the T/R circuit 11 is provided in the ultrasonic diagnostic apparatus 10 will be described, but the T/R circuit 11 may be provided in the ultrasonic probe 20, or may be provided in both of the ultrasonic diagnostic apparatus 10 and the ultrasonic probe 20. The T/R circuit 11 is one example of a transmitter-and-receiver.

The transmitting circuit T supplies a drive signal to the ultrasonic transducer of the ultrasonic probe 20. The configuration of the transmitting circuit T will be described later with reference to FIG. 4. The receiving circuit 112 receives the received signal received by the ultrasonic transducers and performs various processing on the received signal to generate echo data. The configuration of the receiving circuit 112 will be described later with reference to FIG. 4.

The B-mode processing circuit 12 may change the frequency band to be visualized by changing the detection frequency using filtering processing. By using the filtering processing function of the B-mode processing circuit 12, harmonic imaging such as the contrast harmonic imaging (CHI) or the tissue harmonic imaging (THI) is performed.

That is, the B-mode processing circuit 12 may separate the reflected waves from within a subject into which the contrast agent is injected into harmonic data (or sub-frequency data) and fundamental wave data. The harmonic data (or sub-frequency data) corresponds to reflected waves with a harmonic component whose reflection source is the contrast agent (microbubbles or bubbles) in the subject. The fundamental wave data corresponds to reflected waves with a fundamental wave component whose reflection source is tissue in the subject. The B-mode processing circuit 12 generates B-mode data for generating contrast image data based on the reflected wave data (reception signals) of the harmonic component, and generates B-mode data for generating fundamental wave image data based on the reflected wave data (reception signals) with the fundamental wave component.

In the THI by using the filtering processing function of the B-mode processing circuit 12, it is possible to separate harmonic data or sub-frequency data which is reflected wave data (reception signals) of a harmonic component from reflected wave data of the subject. Then, the B-mode processing circuit 12 generates B-mode data for generating tissue image data in which the noise component is removed from the reflected wave data (reception signals) of the harmonic component.

When the CHI or THI harmonic imaging is performed, the B-mode processing circuit 12 may extract the harmonic component by a method different from the method using the above-described filtering. With respect to harmonic imaging, an imaging method called the amplitude modulation (AM) method, the phase modulation (PM) method or the AM-PM method in which the AM method and the PM method are combined is performed. With the AM method, the PM method, and the AM-PM method, ultrasonic transmission with different amplitudes and phases is performed multiple times on the same scanning line.

Thereby, the T/R circuit 11 generates and outputs multiple reflected wave data (reception signals) in each scanning line. The B-mode processing circuit 12 extracts harmonic components by performing addition/subtraction processing according to the modulation method on the multiple reflected wave data (reception signals) of each scanning line. The B-mode processing circuit 12 performs envelope detection processing etc. on the reflected wave data (reception signals) of the harmonic component to generate B-mode data.

For example, when the PM method is performed, the T/R circuit 11 controls the ultrasonic waves having the same amplitude and reversed-phase polarities, for example (−1, 1), to be transmitted twice by each scanning line under a scan sequence set by the processing circuitry 17. The T/R circuit 11 generates a reception signal based on transmission of “−1” and a reception signal based on transmission of “1”. The B-mode processing circuit 12 adds these two reception signals. As a result, the fundamental wave component is removed, and a signal in which the second harmonic component mainly remains is generated. Then, the B-mode processing circuit 12 performs envelope detection processing and the like on such a signal to generate B-mode data using THI or CHI.

Alternatively, for example, in the THI, an imaging method using the second harmonic component and a difference tone component included in the reception signals has been put to practical use. With the imaging method using the difference tone component, transmission ultrasonic waves are transmitted from the ultrasonic probe 20, and the transmission ultrasonic waves have, for example, a composite waveform in which a first fundamental waves with a center frequency “f1” and a second fundamental waves with a center frequency “f2” larger than the center frequency “f1” are combined. Such a composite waveform is a waveform in which a waveform with the first fundamental waves and a waveform with the second fundamental waves which phases being adjusted with each other are combined, such that the difference tone component having the same polarity as the second harmonic component is generated. The T/R circuit 11 transmits the transmission ultrasonic waves of the composite waveform, for example, twice while inverting the phase. In such a case, for example, the B-mode processing circuit 12 removes the fundamental wave component by adding two reception signals, and performs an envelope detection process etc. after extracting a harmonic component in which the difference tone component and the second harmonic component are mainly left.

Under the control of the processing circuitry 17, the Doppler processing circuit 13 frequency-analyzes the phase information from the echo data from the receiving circuit 112, thereby generating data (2D or 2D data) acquired by extracting moving data of moving subject such as average speed, variance, power and the like for multiple points. This data is an example of the raw data, and is generally called “Doppler data”. In the present embodiment, the moving subject is, for example, blood flow, tissue such as heart wall, or contrast agent. The Doppler processing circuit 13 is one example of a Doppler processer.

Under the control of the processing circuitry 17, the image generating circuit 14 generates an ultrasonic image presented in a predetermined luminance range as image data based on the reception signals received by the ultrasonic probe 20. For example, the image generating circuit 14 generates, as an ultrasonic image, a B-mode image in which the intensity of the reflected wave is represented by luminance from the two-dimensional B-mode data generated by the B-mode processing circuit 12. In addition, the image generating circuit 14 generates a color Doppler image from the two-dimensional Doppler data generated by the Doppler processing circuit 13. The color Doppler image includes an average speed image representing moving state information, a variance image, a power image, or a combination image thereof. The image generating circuit 14 is an example of an image generating unit.

In the present embodiment, the image generating circuit 14 generally converts (scan-converts) a scanning line signal sequence of ultrasonic scanning into a scanning line signal sequence of a video format used by a television or the like, and generates ultrasonic image data for display. Specifically, the image generating circuit 14 generates ultrasonic image data for display by performing coordinate conversion according to the ultrasonic scanning mode of the ultrasonic probe 20. The image generating circuit 14 performs various image processes other than the scan conversion. For example, the image generating circuit 14 performs image processing (smoothing processing) for regenerating an average luminance image using multiple image frames after scan conversion, image processing using a differential filter in the image (processing for enhancing edges) and the like. Further, the image generating circuit 14 combines character information of various parameters, scales, body marks, and the like with the ultrasonic image data.

That is, the B-mode data and the Doppler data are the ultrasonic image data before the scan conversion processing. The data generated by the image generating circuit 14 is ultrasonic image data for display after the scan conversion processing. The B-mode data and the Doppler data are also called raw data. The image generating circuit 14 generates two-dimensional ultrasonic image data for display from the two-dimensional ultrasonic image data before the scan conversion processing.

Further, the image generating circuit 14 performs coordinate conversion on the three-dimensional B-mode data generated by the B-mode processing circuit 12, thereby generates three-dimensional B-mode image data. The image generating circuit 14 performs coordinate conversion on the three-dimensional Doppler data generated by the Doppler processing circuit 13, thereby generates three-dimensional Doppler image data. The image generating circuit 14 generates “three-dimensional B-mode image data or three-dimensional Doppler image data” as “three-dimensional ultrasonic image data (volume data)”.

Further, the image generating circuit 14 performs a rendering processing on the volume data to generate various two-dimensional image data for displaying the volume data on the display 40. The image generating circuit 14 performs a processing of generating a multi planer reconstruction (MPR) image data from the volume data by performing, for example, an MPR processing that is one of the rendering processing. Further, the image generating circuit 14 performs, for example, volume rendering (VR) processing for generating two-dimensional image data reflecting three-dimensional data that is one of the rendering processing.

The image memory 15 includes multiple memory cells in one frame in two axial directions, and includes a two-dimensional memory which is a memory provided with multiple frames. The two-dimensional memory as the image memory 15 stores one frame or an ultrasonic image of the multiple frames generated by the image generating circuit 14 as two-dimensional image data under the control of the processing circuitry 17. The image memory 15 is an example of a storage.

Under the control of the processing circuitry 17, the image generating circuit 14, if necessary, performs three-dimensional reconstruction for performing an interpolation processing on the ultrasonic images arranged in the two-dimensional memory as the image memory 15, thereby generates an ultrasonic image as volume data in the three-dimensional memory as the image memory 15. A known technique is used as the interpolation processing.

The image memory 15 may include a three-dimensional memory which is a memory having multiple memory cells in three axis directions (X-axis, Y-axis, and Z-axis directions).

The three-dimensional memory as the image memory 15 stores the ultrasonic image generated by the image generating circuit 14 as volume data under the control of the processing circuitry 17.

The network interface 16 implements various information communication protocols according to the network form. The network interface 16 connects the ultrasonic diagnostic apparatus 10 and other devices such as the external medical image managing apparatus 60 and the medical image processing apparatus 70 according to these various protocols. An electrical connection or the like via an electronic network is applied to this connection. In the present embodiment, the electronic network means an entire information communication network using telecommunications technology. The electronic network includes a wired/wireless hospital backbone local area network (LAN) and the Internet network, as well as a telephone communication line network, an optical fiber communication network, a cable communication network, a satellite communication network, or the like.

Further, the network interface 16 may implement various protocols for non-contact wireless communication. In this case, the ultrasonic diagnostic apparatus 10 can directly transmit/receive data to/from the ultrasonic probe 20, for example, without going through the network. The network interface 16 is one example of a network connector.

The processing circuitry 17 may mean a processor such as a dedicated or general-purpose CPU (central processing unit), an MPU (microprocessor unit), a GPU (Graphics Processing Unit), or the like. The processing circuitry 17 may mean an ASIC, a programmable logic device, or the like. The programmable logic device is, for example, a simple programmable logic device (SPLD), a complex programmable logic device (CPLD), and a field programmable gate array (FPGA).

Further, the processing circuitry 17 may be constituted by a single circuit or a combination of independent circuit elements. In the latter case, the main memory 18 may be provided individually for each circuit element, or a single main memory 18 may store programs corresponding to the functions of the circuit elements. The processing circuitry 17 is one example of a processor.

The main memory 18 is constituted by a semiconductor memory element such as a random-access memory (RAM), a flash memory, a hard disk, an optical disk, or the like. The main memory 18 may be constituted by a portable medium such as a universal serial bus (USB) memory and a digital video disk (DVD). The main memory 18 stores various processing programs (including an operating system (OS) and the like besides the application program) used in the processing circuitry 17 and data necessary for executing the programs. In addition, the OS may include a graphical user interface (GUI) which allows the operator to frequently use graphics to display information on the display 40 to the operator and can perform basic operations by the input interface 30. The main memory 18 is one example of a storage.

The ultrasonic probe 20 includes microscopic transducers (piezoelectric elements) on the front surface portion, and transmits and receives ultrasonic waves to a region including a scan target, for example, a region including a lumen. Each transducer is an electroacoustic transducer, and has a function of converting electric pulses into ultrasonic pulses at the time of transmission and converting reflected waves to electric signals (reception signals) at the time of reception. The ultrasonic probe 20 is configured to be small and lightweight, and is connected to the ultrasonic diagnostic apparatus 10 via a cable (or wireless communication).

The ultrasonic probe 20 is classified into types such as a linear type, a convex type, a sector type, etc. depending on differences in scanning system. Further, the ultrasonic probe 20 is classified into a 1D array probe in which transducers are arrayed in a one-dimensional (1D) manner in the azimuth direction, and a 2D array probe in which transducers are arrayed in a two-dimensional (2D) manner in the azimuth direction and in the elevation direction, depending on the array arrangement dimension. The 1D array probe includes a probe in which a small number of transducers are arranged in the elevation direction.

In the present embodiment, when a three-dimensional (3D) scan, that is, a volume scan is executed, the 2D array probe having a scan type such as the linear type, the convex type, the sector type, or the like is used as the ultrasonic probe 20. Alternatively, when the volume scan is executed, the 1D probe having a scan type such as the linear type, the convex type, the sector type and the like and having a mechanism that mechanically oscillates in the elevation direction is used as the ultrasonic probe 20. The latter probe is also called a mechanical 4D probe.

The input interface 30 includes an input device operable by an operator, and a circuit for inputting a signal from the input device. The input device may be a trackball, a switch, a mouse, a keyboard, a touch pad for performing an input operation by touching an operation surface, a touch screen in which a display screen and a touch pad are integrated, a non-contact input circuit using an optical sensor, an audio input circuit, and the like. When the input device is operated by the operator, the input interface 30 generates an input signal corresponding to the operation and outputs it to the processing circuitry 17.

The input interface 30 may further include an adjustment switch for adjusting a frequency characteristic of a reception filter to be described later. The input interface 30 is one example of an input unit.

The display 40 is constituted by a general display output device such as a liquid crystal display or an organic light emitting diode (OLED) display. The display 40 displays various kinds of information under the control of the processing circuitry 17. The display 40 is one example of a display unit.

FIG. 1 shows the medical image managing apparatus 60 and the medical image processing apparatus 70 which are external devices of the ultrasonic diagnostic apparatus 10. The medical image managing apparatus 60 is, for example, a digital imaging and communications in medicine (DICOM) server, and is connected to a device such as the ultrasonic diagnostic apparatus 10 such that data can be transmitted and received via the network N. The medical image managing apparatus 60 manages a medical image such as an ultrasonic image generated by the ultrasonic diagnostic apparatus 10 as the DICOM file.

The medical image processing apparatus 70 is connected to devices such as the ultrasonic diagnostic apparatus 10 and the medical image managing apparatus 60 such that data is transmitted and received via the network N. An Example of the medical image processing apparatus 70 includes a workstation that performs various image processing on the ultrasonic image generated by the ultrasonic diagnostic apparatus 10 and a portable information processing terminal such as a tablet terminal. It should be noted that the medical image processing apparatus 70 is an offline apparatus and may be an apparatus capable of reading an ultrasonic image generated by the ultrasonic diagnostic apparatus 10 via a portable storage medium.

Subsequently, the concept of the configuration and function of the receiving circuit 112 provided in the T/R circuit 11 will be described with reference to FIGS. 2 and 3.

The receiving circuit 112 provided in the T/R circuit 11 has a frequency characteristic analysis circuit (for example, “frequency characteristic analysis circuit 57” shown in FIG. 4), a filter setting circuit (for example, “filter setting circuit 58” shown in FIG. 4), and a filter processing circuit (for example, “filter processing circuit 56” shown in FIG. 4). The frequency characteristic analysis circuit perform a frequency analysis on a reception signal of a predetermined depth based on the reception signals of the ultrasonic wave from the ultrasonic probe 20, and acquires the frequency characteristic. The filter setting circuit sets a reception filter that corrects the frequency characteristic at a predetermined depth such that the frequency characteristic at a predetermined depth acquired by the frequency characteristic analysis circuit shows the predetermined frequency characteristic. The filter processing circuit applies the reception filter set by the filter setting circuit to the reception signal of a predetermined depth such that feedback is performed. For example, the frequency characteristic analysis circuit perform the frequency-analysis on a reception signal in each region of interest according to the depth and acquires a frequency characteristic in each region of interest. The filter setting circuit sets a reception filter that corrects the frequency characteristic of each region of interest in each region of interest such that the frequency characteristic of each region of interest acquired by the frequency characteristic analysis circuit shows a predetermined frequency characteristic. The filter processing circuit applies the reception filter in each region of interest set by the filter setting circuit to the reception signal in each region of interest such that feedback is performed.

That is, the filter setting circuit sets a reception filter that is variable according to the depth and that corrects the reception signal so as to exhibit a frequency characteristic having a substantially flat bandwidth over a wide range. It should be noted that “substantially flat” means that the absolute value of the slope of the tangent line formed by each point on the waveform is equal to or smaller than a threshold value, that is, it is rather level. In addition, the reception filter may correct the reception signal so as to exhibit symmetrical (for example, Gaussian function) frequency characteristic on the low frequency side and the high frequency side with respect to the center frequency.

FIG. 2 is a conceptual diagram for explaining a target frequency characteristic having the substantially flat bandwidth close to a designed frequency characteristic.

The left side of each of FIGS. 2A and 2B shows a designed frequency characteristic. The center of each of FIGS. 2A and 2B shows a frequency characteristic based on a reception signal in the region of interest in the clinic practice. The right side of each of FIGS. 2A and 2B shows a target frequency characteristic having a frequency characteristic of a substantially flat bandwidth close to a substantially flat bandwidth in design. FIG. 2A shows a frequency characteristic particularly in a shallow part when ultrasonic attenuation is small and high frequency is dominant in the clinical practice. FIG. 2B shows a frequency characteristic particularly in a deep part when ultrasonic attenuation is large and low frequency is dominant in clinical practice.

The filter setting circuit calculates a target frequency characteristic shown on the right side based on a designed frequency characteristic shown on the left side of FIG. 2A and a clinical frequency characteristic shown at the center. For example, the target frequency characteristic has a characteristic that becomes substantially flat in a wide band.

As shown on the right side of FIG. 2A, the target frequency characteristic has a wide band on the high frequency side, and the intensity is not biased between the high frequency side and the low frequency side. The reception filter set by the filter setting circuit shapes the waveform of the clinical frequency characteristic such that the clinical frequency characteristic shown at the center of FIG. 2A is close to a substantially flat bandwidth shown on the left side.

On the other hand, the filter setting circuit calculates a target frequency characteristic shown on the right side based on a designed frequency characteristic shown on the left side of FIG. 2B and a clinical frequency characteristic shown at the center. For example, the target frequency characteristic has a characteristic that becomes substantially flat in a wide band.

As shown on the right side of FIG. 2B, the target frequency characteristic has a wide band on the low frequency side, and the intensity is not biased between the high frequency side and the low frequency side. The reception filter set by the filter setting circuit shapes the waveform of the clinical frequency characteristic such that the clinical frequency characteristic shown at the center of FIG. 2B is close to a substantially flat bandwidth shown on the left side.

Each of FIGS. 3A to 3E is a conceptual diagram for explaining a method of setting a reception filter.

FIG. 3A shows frequency characteristic acquired by frequency-analyzing a reception signal in a region of interest corresponding to the depth among reception signals of one frame. FIG. 3A has the same waveform as shown at the center of FIG. 2B. FIG. 3B shows the gravity center calculated based on the frequency characteristic shown in FIG. 3A in a broken line. FIG. 3C shows a target signal strength calculated from the frequency characteristic shown in FIG. 3A.

FIG. 3D shows, in a thick solid line, a frequency characteristic having the signal strength shown in FIG. 3C and having a substantially flat bandwidth close to the target substantially flat bandwidth. FIG. 3E shows, in a thick solid line, in which the frequency characteristic of FIG. 3A is set to indicate the target frequency characteristic of FIG. 3D.

As shown in FIGS. 3A to 3E, the filter setting circuit sets a reception filter, thereby brings the substantially flat bandwidth indicated by the frequency characteristic of the reception signal in each region of interest in the clinical practice closer to the substantially flat bandwidth indicated by the designed frequency characteristic.

The reception signals for acquiring the frequency characteristic may be RF signals or I/Q signals. That is, the beamforming method may be RF beamforming or I/Q beamforming. With the RF beamforming method, the RF signals are delayed and added, subjected to quadrature detection (demodulation), converted into I/Q signals each including an “I (In-phase)” signal and a “Q (Quadrature-phase)” signal, and an ultrasonic image is generated. With the I/Q beamforming method, the RF signals are subjected to quadrature detection, converted to an I/Q basebands, delayed and added, and an ultrasonic image is generated. Hereinafter, a case where the reception signals for acquiring frequency characteristic are the I/Q signals, that is, a case where the I/Q beamforming is adopted, will be described as an example unless otherwise specified.

Subsequently, a specific configuration and functions of the receiving circuit 112 provided in the T/R circuit 11 will be described with reference to FIGS. 4 to 12.

FIG. 4 is a block diagram showing the configuration of the T/R circuit 11.

FIG. 4 shows a transmitting circuit T provided in the T/R circuit 11 and a receiving circuit 112. The transmitting circuit T has a pulse generating circuit T1, a transmission delay circuit T2, a drive circuit (e.g., pulsar), and supplies a drive signal to ultrasonic transducers. The pulse generating circuit T1 repeatedly generates rate pulses for forming transmission ultrasonic waves at a predetermined rate frequency. The transmission delay circuit T2 converges the ultrasonic waves generated from the ultrasonic transducer of the ultrasonic probe 20 into a beam shape, and gives a delay time of each piezoelectric transducer necessary for determining the transmission directivity to each rate pulse generated by the pulse generating circuit T1. The transmission delay circuit T2 arbitrarily adjusts the transmission direction of the ultrasonic beam transmitted from a piezoelectric transducer surface by changing the delay time given to each rate pulse. The drive circuit T3 applies a drive pulse to the ultrasonic transducer at a timing based on the rate pulse.

The receiving circuit 112 includes an amplifier 51, an analog to digital (A/D) conversion circuit 52, a quadrature detection circuit 53, a reception delay circuit 54, an addition circuit 55, a filter processing circuit 56, a frequency characteristic analysis circuit 57, and a filter setting circuit 58.

The amplifier 51 has a function of amplifying signals received from the ultrasonic probe 20 for each channel and performing a gain correction processing under the control of the processing circuitry 17. The amplifier 51 can improve the image quality of the ultrasonic image by controlling the gain.

The A/D conversion circuit 52 has a function of subjecting the gain-corrected reception signals, which is the output of the amplifier 51, to A/D conversion for each channel under the control of the processing circuitry 17.

The quadrature detection circuit 53 has a function of performing quadrature detection on RF signals that are reception signals and converting the RF signals into I/Q signals each including an “I” signal and a “Q” signal for each channel.

The reception delay circuit 54 has a function of giving, for each channel, a delay time necessary for determining the reception directivity to the I/Q signals output from the quadrature detection circuit 53 under the control of the processing circuitry 17. The reception delay circuit 54 can improve the image quality of the ultrasonic image by controlling the reception delay curve given to the I/Q signals.

The addition circuit 55 has a function of performing phase rotation and weighting control (apodization) for each channel on the I/Q signals output from the reception delay circuit 54 to acquire the I/Q signals, thereby adding the acquired I/Q signals and generating beam data of the I/Q signals. By the addition processing of the addition circuit 55, a reflection component from a direction corresponding to the reception directivity of the reception signals is emphasized.

The filter processing circuit 56 has a function of applying an arbitrary complex reception filter to the I/Q signals output from the addition circuit 55 under the control of the processing circuitry 17, and a function of outputting the I/Q signals to which the complex reception filter has been applied to the B-mode processing circuit 12 and the Doppler processing circuit 13. The filter processing circuit 56 is an example of a filter processor.

As described above, the image quality of the ultrasonic image can be improved to some extent by the gain control by the amplifier 51 and the control of the reception delay curve by the reception delay circuit 54. However, since the ultrasonic attenuation changes for each individual and of each depth, it is difficult to optimize the image quality of the ultrasonic wave only by controlling those. Therefore, the receiving circuit 112 provided in the T/R circuit 11 includes the frequency characteristic analysis circuit 57 and the filter setting circuit 58.

The frequency characteristic analysis circuit 57 has a function of performing a frequency analysis on an I/Q signal in each region of interest according to the depth under the control of the processing circuitry 17 based on the I/Q signals output from the addition circuit 55, thereby acquiring a frequency characteristic. For example, the frequency characteristic analysis circuit 57 can perform frequency analysis by performing a fast Fourier transform (FFT) on the I/Q signal in each region of interest. The frequency characteristic analysis circuit 57 is one example of a frequency characteristic analyzer.

The filter setting circuit 58 sets, under the control of the processing circuitry 17, a complex reception filter in each region of interest, which is output from the frequency characteristic analysis circuit 57, such that the frequency characteristic of each region of interest shows a predetermined frequency characteristic. The filter coefficient of the complex reception filter is a complex coefficient including a real part and an imaginary part. In the case of the I/Q beamforming, when the waveform of the I/Q signal in each region of interest slightly changes the frequency of the waveform of the RF signal, the modulated signal can be treated as a complex amplitude. The filter setting circuit 58 is one example of a filter setting unit. Under the control of the processing circuitry 17, the filter processing circuit 56 includes a function of applying the complex reception filter of each region of interest output from the filter setting circuit 58 to the I/Q signals output from the addition circuit 55 such that feedback is performed, and a function of outputting the I/Q signals to which the complex reception filter has been applied to the B-mode processing circuit 12 or the Doppler processing circuit 13 as baseband data, in addition to the function described above.

sequently, an operation of the ultrasonic diagnostic apparatus 10 will be described.

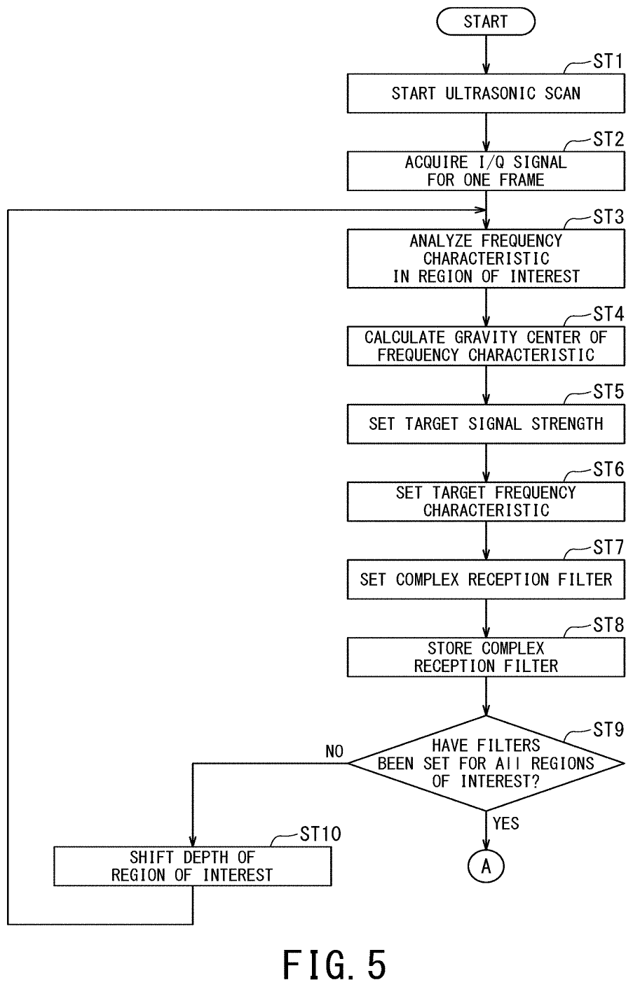

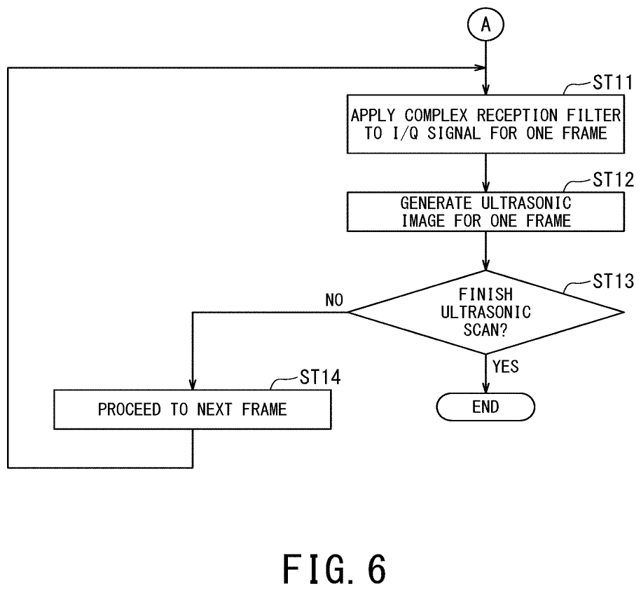

Each of FIGS. 5 and 6 is a diagram showing the operation of the ultrasonic diagnostic apparatus 10 as a flowchart. In FIGS. 5 and 6, reference numerals with numbers attached to “ST” indicate respective steps in the flowchart. In FIGS. 5 and 6, the case of I/Q beamforming, that is, the case where the reception filter is a complex reception filter will be described as an example.

As shown in FIG. 5, the processing circuitry 17 of the ultrasonic diagnostic apparatus 10 controls the T/R circuit 11 and the like to start an ultrasonic scan using the ultrasonic probe 20 (step ST1).

The frequency characteristic analysis circuit 57 acquires I/Q signals for one frame, which are the output of the addition circuit 55 (step ST2). The frequency characteristic analysis circuit 57 performs the frequency analysis on an I/Q signal in the region of interest corresponding to the depth among the I/Q signals for one frame acquired in step ST2 to acquire frequency characteristic (step ST3).

filter setting circuit 58 sets a complex reception filter for correcting the waveform of the frequency characteristic of the region of interest such that the frequency characteristic of the region of interest acquired in step ST3 shows a predetermined frequency characteristic specifically, it is based on steps ST4 to ST8 described later.

The filter setting circuit 58 calculates the gravity center from the frequency characteristic of the region of interest acquired in step ST3 (step ST4).

The filter setting circuit 58 sets a target signal strength (step ST5). The filter setting circuit 58 sets a target frequency characteristic, based on the waveform of the frequency characteristic acquired in step ST3, the gravity center set in step ST4, the target signal strength set in step ST5, and the substantially flat bandwidth indicated by the designed frequency characteristic (step ST6. The designed frequency characteristic is set in advance before the ultrasonic scan is started, or is optimized according to the acquired frequency characteristic.

For example, the filter setting circuit 58 acquires a reception filter coefficient having a target signal strength near the position of the gravity center and having a substantially flat bandwidth close to a substantially flat bandwidth in design. The receive filter coefficient is based on a waveform of clinical frequency characteristic acquired in step ST3.

The filter setting circuit 58 sets a complex reception filter for the region of interest, and the complex reception filter shapes the waveform such that the frequency characteristic of the I/Q signal in the region of interest acquired in step ST3 indicates the target frequency characteristic set in step ST6 (step ST7), and stores the complex reception filter of the region of interest in the main memory 18 (step ST8 ). The operator may adjust the frequency characteristic of the set complex reception filter via the input interface 30 (adjustment switch).

Each of FIGS. 7A and 7B is a diagram showing a complex reception filter of the region of interest.

FIG. 7A shows a frequency characteristic of an I/Q signal in the region of interest, a target frequency characteristic of the region of interest, and a frequency characteristic of a complex reception filter of the region of interest. A complex reception filter for shaping the waveform is set for each region of interest such that the frequency characteristic of the I/Q signal in the region of interest indicates the target frequency characteristic.

In the case of I/Q beamforming, when the waveform of the I/Q signal in each region of interest slightly changes the frequency of the waveform of the RF signal, the modulated signal can be treated as a complex amplitude. The filter coefficient of the complex reception filter is a complex coefficient including a real part and an imaginary part.

Returning to the description of FIG. 5, the filter setting circuit 58 determines whether or not complex reception filters have been set at all depths, that is, in all regions of interest (step ST9). If it is determined as “NO” in step ST9, that is, if it is determined that the complex reception filters are not set in all the regions of interest, the frequency characteristic analysis circuit 57 shifts the depth of the region of interest (step ST10), and frequency-analyzes the shifted I/Q signal in the region of interest based on the I/Q signal for one frame acquired in step ST2 to acquire frequency characteristic (step ST3).

On the other hand, if it is determined as “YES” in step ST9, that is, if it is determined that the complex reception filters have been set in all the regions of interest, the processing proceeds to the steps in FIG. 6.

Each of FIGS. 8A and 8B is a diagram showing a complex reception filter of each region of interest. FIG. 8 shows a complex reception filter of each region of interest when divided into eight in the depth direction.

FIG. 8A shows the filter coefficients of the real part components in each region of interest, that is, at each depth. FIG. 8B shows the filter coefficient of the imaginary part component in each region of interest, that is, at each depth. As shown in FIGS. 8A and 8B, appropriate filter coefficients are calculated for the real part component and the imaginary part component of each region of interest.

Returning to the description of FIG. 6, the filter processing circuit 56 applies the complex reception filter in each region of interest registered in step ST8 to the I/Q signals of one frame output from the addition circuit 55 such that feedback is performed (step ST11).

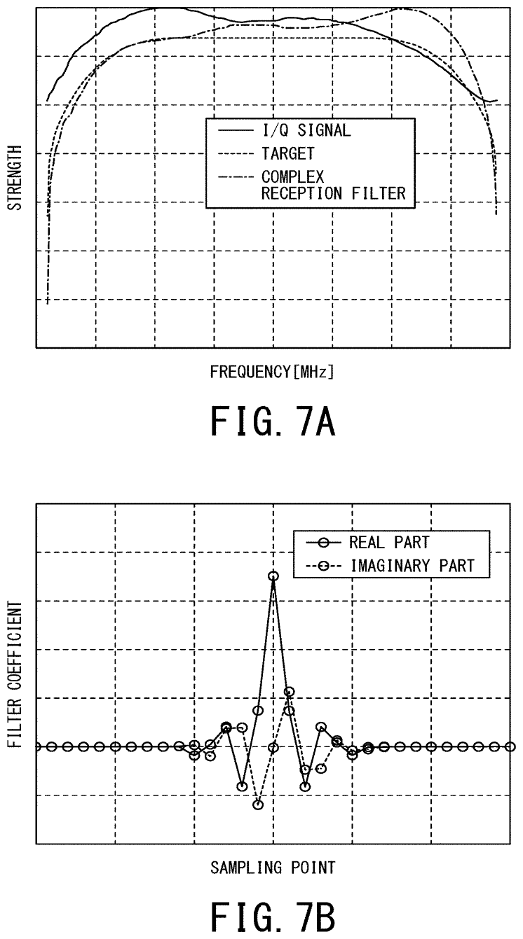

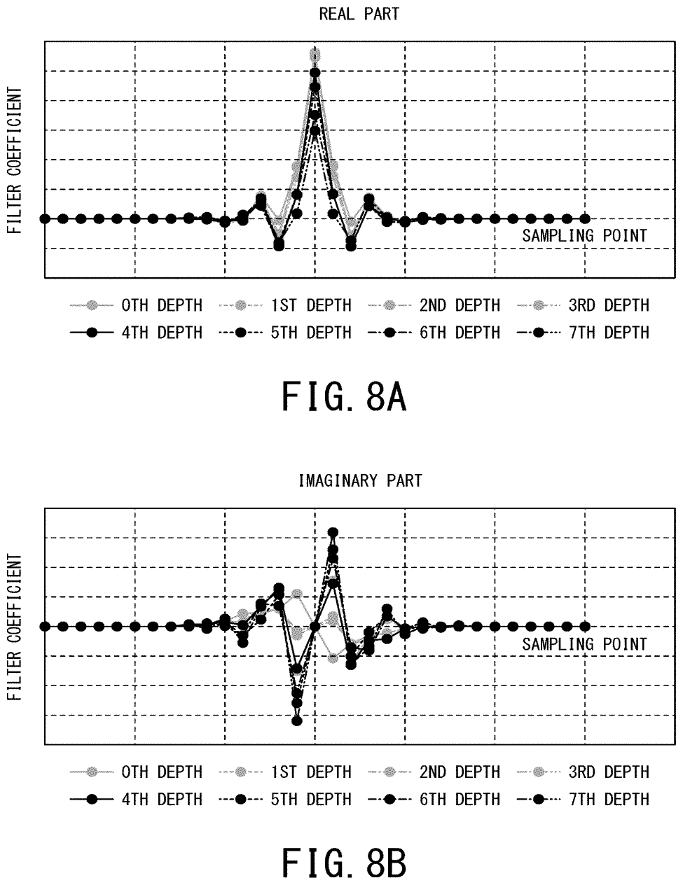

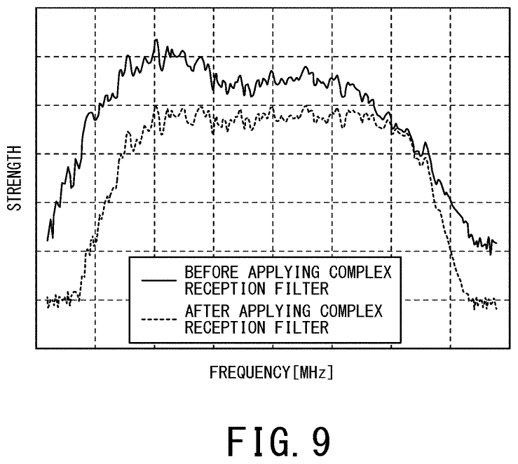

FIG. 9 is a diagram showing, as a frequency characteristic, an effect acquired when a complex reception filter is applied to the I/Q signal in a predetermined region of interest.

FIG. 9 shows frequency characteristic before a complex reception filter is applied to I/Q signals for one frame. FIG. 9 also shows frequency characteristic after applying a complex reception filter to I/Q signal in a predetermined region of interest among I/Q signals for one frame. The complex reception filter in each region of interest registered in step ST8 is fed back and applied to the I/Q signals for one clinical frame, which is the output of the addition circuit 55. As a result, the frequency characteristic of the I/Q signals for one clinical frame are corrected to the target frequency characteristic, and a substantially flat bandwidth is expanded.

Returning to the description of FIG. 6, the B-mode processing circuit 12 (or Doppler processing circuit 13) and the image generating circuit 14 generate an ultrasonic image for one frame based on the I/Q signals in the entire range to which the complex reception filter has been applied in step ST11 (step ST12).

Each of FIGS. 10A and 10B is a diagram showing an effect acquired when a complex reception filter is applied to the I/Q signal in a predetermined region of interest as an ultrasonic image (for example, a B-mode image). The imaging target (part) of the B-mode image shown in each of FIGS. 10A and 10B is a kidney.

FIG. 10A shows a B-mode image before applying a complex reception filter to I/Q signals for one frame. FIG. 10B shows a B-mode image after applying a complex reception filter to an I/Q signal in a predetermined region of interest, for example, a region of interest R, of I/Q signals for one frame.

The B-mode image region shown in FIG. 10A is compared with the B-mode image region shown in FIG. 10B. According to the B-mode image region shown in FIG. 10B, the distance resolution of the structure in the region of interest R of the kidney is improved. As a result, the image quality is optimized, and it is possible to more clearly recognize the area of interest R.

Returning to the description of FIG. 6, the processing circuitry 17 determines whether to finish the ultrasonic scan started in step ST1 (step ST13). For example, the processing circuitry 17 determines whether or not to finish the ultrasonic scan by a finish operation by the operator via the input interface 30. If it is determined as “NO” in step ST13, that is, if it is determined that the ultrasonic scan started in step ST1 is not to be finished, proceed to the next frame (step ST14), and the filter processing circuit 56 applies the coefficients of the complex reception filter registered in step ST8 to the I/Q signals of the next one frame such that feedback is performed (step ST11).

On the other hand, if it is determined as “YES” in step

ST13, that is, if it is determined that the ultrasonic scan started in step ST1 is to be finished, the processing circuit 17 of the ultrasonic diagnostic apparatus 10 controls the T/R circuit 11 and the like, thereby finishes the ultrasonic scan using the ultrasonic probe 20.

The case where, in the ultrasonic scan for the same patient and the same imaging part, the complex reception filter set and registered once is applied to I/Q signals of frames generated thereafter has been described with reference to FIGS. 5 and 6. In other words, the same complex reception filter is used during a series of ultrasonic examinations for scanning the same imaging part. However, it is not limited to that case. For example, the complex reception filter may be set every time in each frame, or may be set at fixed intervals. The necessity of setting the complex reception filter may be switched of each frame according to the movement of the ultrasonic probe 20.

In this case, the frequency characteristic analysis circuit 57 determines whether or not the change in the value indicating the scan cross-section is equal to or greater than the threshold value. Then, the frequency characteristic analysis circuit 57 may perform frequency analysis of the I/Q signal again, or may return to the complex reception filter originally set in the apparatus as a fixed value when the change in the value indicating the scan cross-section is equal to or greater than the threshold value and when there is almost no change in the value indicating the scan cross-section, that is, the change less than the threshold value. The value indicating the scan cross-section is a value indicating at least one of the positions and angles of the ultrasonic probe 20 corresponding to the scan cross-section.

Alternatively, the value indicating the scan cross-section is the luminance value of the ultrasonic image corresponding to the scan cross-section. The luminance value of the ultrasonic image refers to a variation in the average luminance value, the maximum luminance value, the minimum luminance value, or the luminance value in pixels constituting the ultrasonic image (or a region of interest thereof).

That is, the complex reception filter is not reset while the position and angle of the ultrasonic probe 20 change to some extent between frames. This is because the change in the value indicating the scan cross-section exceeds the threshold value. Alternatively, the complex reception filter is not reset while the average luminance values of the pixels constituting the ultrasonic image (or the region of interest thereof) change to some extent between frames. This is because the change in the value indicating the scan cross-section exceeds the threshold value. The change in the value indicating the scan cross-section may be based on data acquired by an acceleration sensor (not shown) or a magnetic sensor (not shown). The acceleration sensor can measure the angle of the ultrasonic probe 20 provided in the ultrasonic probe 20. The magnetic sensor can generate a magnetic field to measure the position and angle of the ultrasonic probe 20. Alternatively, when the sensor is not used, a change in the value indicating the scan cross-section may be detected from the time change of the image information.

Further, the complex reception filter set in the past and the value indicating the scan cross-section (e.g., the position of the ultrasonic probe 20) may be registered in the main memory 18. In this case, if it is determined that a scan cross-section of the same position has been scanned in a series of ultrasonic examinations in the past, the filter processing circuit 56 acquires and uses a complex reception filter corresponding to the scan cross-section from the main memory 18. Thereby, the load for setting the complex reception filter is reduced.

Designed frequency characteristic is distorted due to the amount of ultrasonic attenuation from the designed frequency band. However, according to the ultrasonic diagnostic apparatus 10, it is possible to correct instantaneously (or almost in real time) the distortion. As a result, it is possible to suppress image quality degradation due to ultrasonic attenuation, thereby provide a high-quality ultrasonic image.

2. First Modification

The filter setting circuit 58 is not limited to acquiring one target frequency characteristic from the frequency characteristic of the I/Q signal in each region of interest in order to set the reception filter. For example, the filter setting circuit 58 compounds multiple frequency components, that is, generates an ultrasonic image by frequency compounding. In this case, a complex reception filter is set for each frequency component set in each region of interest. The filter setting circuit 58 acquires a target frequency characteristic on the low frequency side and a target frequency characteristic on the high frequency side from the frequency characteristic of the I/Q signal in each region of interest.

In this case, the filter processing circuit 56 corrects the clinical frequency characteristic so as to show the frequency characteristic of each target. Then, the image generating circuit 14 synthesizes the ultrasonic images acquired with the frequency characteristic of each target. As a result, it is effective in improving the contrast resolution and improving the uniformity of the resulting image.

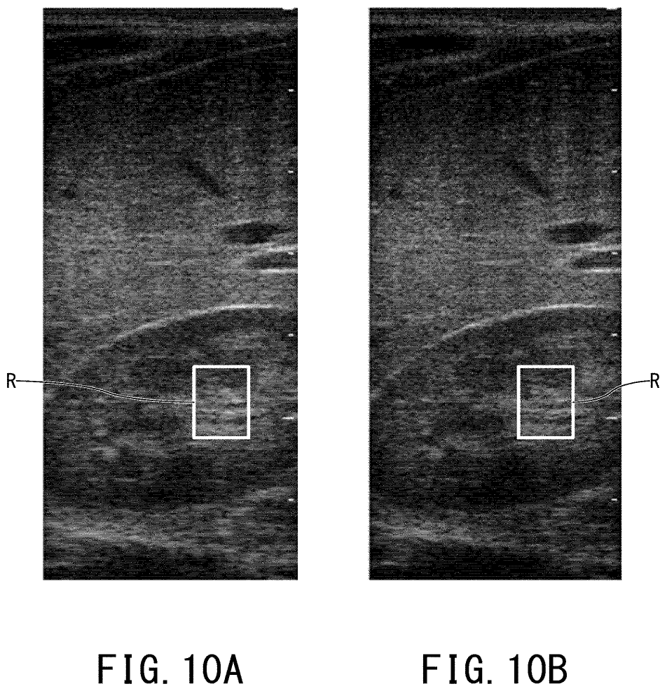

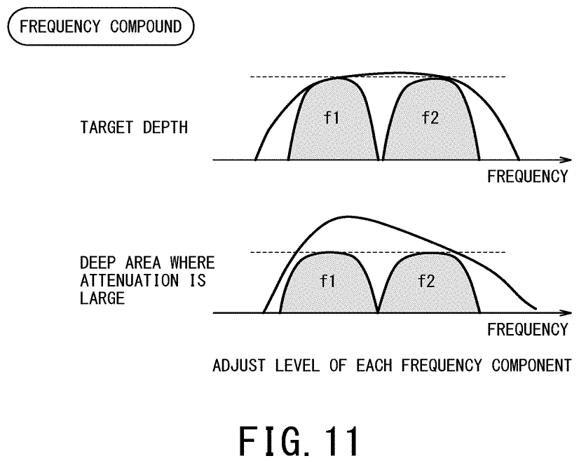

FIG. 11 is a diagram for explaining the frequency compound.

The upper part of FIG. 11 shows the target, that is, the target frequency characteristic (center frequency f1) on the low frequency side and the target frequency characteristic (center frequency f2) on the high frequency side in the region of interest at the depth of the imaging target. The lower part of FIG. 11 shows a target frequency characteristic on the low frequency side (center frequency f1) and a target frequency characteristic on the high frequency side (center frequency f2) in a deep region of interest where ultrasonic attenuation is large. When frequency compounding is performed, as shown in FIG. 11, it is preferable that the level, that is, the intensity is matched between the target frequency characteristic on the low frequency side and the target frequency characteristic on the high frequency side.

3. Second Modification

When performing the frequency analysis on the I/Q signal in each region of interest according to the depth, the frequency characteristic analysis circuit 57 may set each region of interest to include the center position in the scanning direction in the image region. This is because the desired area is often near the center of the image region. However, it is not limited to that case. For example, the frequency characteristic analysis circuit 57 can also set multiple regions of interest of the same depth and perform frequency analysis in a region of interest selected from the set multiple regions of interest.

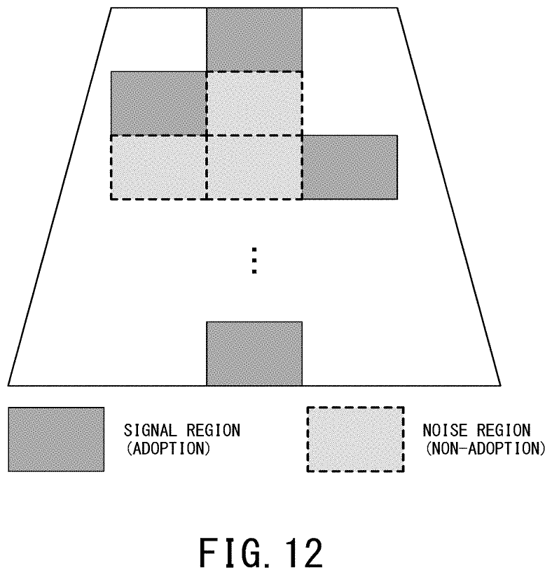

FIG. 12 is a diagram showing a method of selecting a predetermined region of interest from the set multiple regions of interest of the same depth.

FIG. 12 simulates the image region of the B-mode image. Multiple regions of interest are set of the same depth along the scanning direction (the horizontal direction in FIG. 12). Then, the frequency characteristic analysis circuit 57 performs noise determination on the multiple regions of interest of the same depth. The frequency characteristic analysis circuit 57 determines whether the signal to noise (SN) is higher than a threshold value for the multiple regions of interest of the same depth in order from the center position in the scanning direction to the outer position. For example, the frequency characteristic analysis circuit 57 performs noise determination in the region of interest at the center position in the scanning direction at the shallowest part of the image region. Thereafter, the frequency characteristic analysis circuit 57 determines that the region of interest is a signal region, and performs a frequency analysis in the region of interest.

Subsequently, the frequency characteristic analysis circuit 57 performs noise determination in the region of interest at the center position in the scanning direction in the second shallowest part of the image region. Thereafter, the frequency characteristic analysis circuit 57 determines that the region of interest is a noise region. Subsequently, in the second shallowest part of the image region, the frequency characteristic analysis circuit 57 performs a noise determination in the region of interest on the left of the center position. Thereafter, the frequency characteristic analysis circuit 57 determines that the region of interest is the signal region, and performs frequency analysis in the region of interest.

Subsequently, the frequency characteristic analysis circuit 57 performs a noise determination in the region of interest at the center position in the scanning direction in the third shallowest part of the image region. Thereafter, the frequency characteristic analysis circuit 57 determines that the region of interest is the noise region.

Subsequently, the frequency characteristic analysis circuit 57 performs noise determination in the region of interest on the left of the center position in the third shallowest part of the image region. Thereafter, the frequency characteristic analysis circuit 57 determines that the region of interest is the noise region. Subsequently, the frequency characteristic analysis circuit 57 performs noise determination in the region of interest on the right of the center position in the third shallowest part of the image region. Thereafter, the frequency characteristic analysis circuit 57 determines that the region of interest is the signal region, and performs frequency analysis in the region of interest.

In the present embodiment, none of the regions of interest at a certain depth may be the signal region. In this case, a dynamic filter preset in the apparatus may be used, one reception filter set at one shallower or deeper depth may be used, or a representative value (e.g., an average value) of two reception filters set at one shallower and deeper depth may be interpolated and used as the reception filter of the depth.

As described above, according to the ultrasonic diagnostic apparatus 10, by controlling the complex reception filter according to the depth, deterioration of image quality due to ultrasonic attenuation can be suppressed. Thereby, it is possible to provide a high-quality ultrasonic image.

4. Ultrasonic Diagnostic Apparatus According to Second Embodiment

The first embodiment described above realizes the provision of the high-quality ultrasonic image by controlling the receiving side of the ultrasonic wave, that is, the complex receiving filter according to the depth. However, the provision of the high-quality ultrasonic image may be realized by controlling the transmission side of the ultrasonic wave, that is, the transmission frequency according to a degree of beam penetration to a deep portion. A case will be described below as an ultrasonic diagnostic apparatus according to the second embodiment.

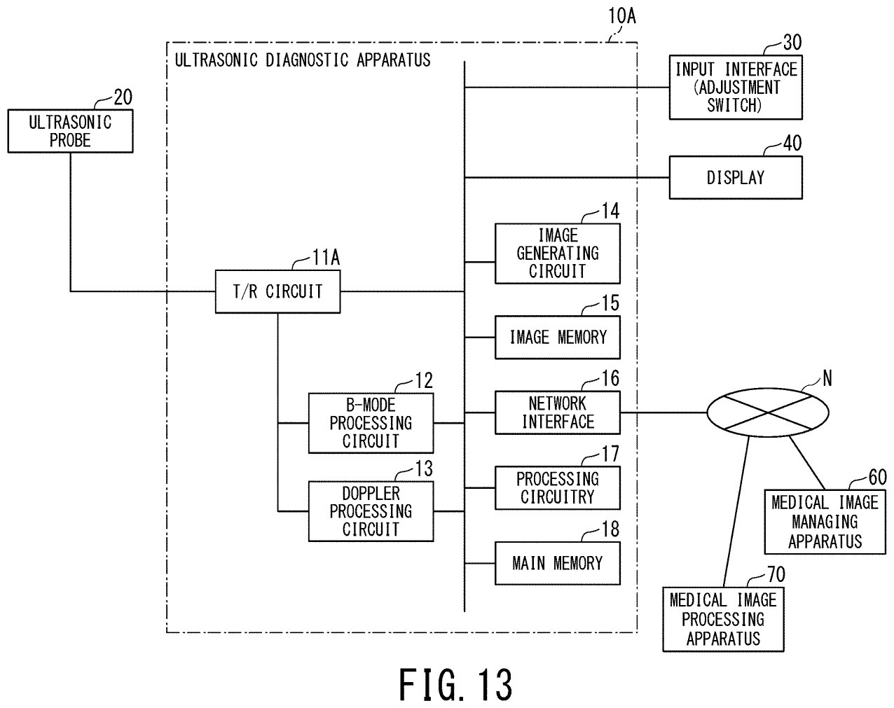

FIG. 13 is a schematic view showing a configuration of the ultrasonic diagnostic apparatus according to a second embodiment.

FIG. 13 shows the ultrasonic diagnostic apparatus 10A according to the second embodiment. Further, FIG. 13 shows an ultrasonic probe 20, an input interface 30, and a display 40. An apparatus in which at least one of an ultrasonic probe 20, an input interface 30, and a display 40 is added to the ultrasonic diagnostic apparatus 10A may be referred to as an “ultrasonic diagnostic apparatus”. In the following description, a case where the ultrasonic probe 20, the input interface 30, and the display 40 that are all provided outside the ultrasonic diagnostic apparatus 10A will be described.

The ultrasonic diagnostic apparatus 10A includes a T/R circuit 11A, a B-mode processing circuit 12, a Doppler processing circuit 13, an image generating circuit 14, an image memory 15, a network interface 16, processing circuitry 17, and a main memory 18. The circuits 11A, 12 to 14 are configured by an integrated circuit for a specific application or the like. However, the present invention is not limited to this case, and all or a part of the functions of the circuits 11A, 12 to 14 may be realized by the processing circuitry 17 executing the program.

In FIG. 13, the same parts as those shown in FIG. 1 are designated by the same reference numerals, and the description thereof will be omitted.

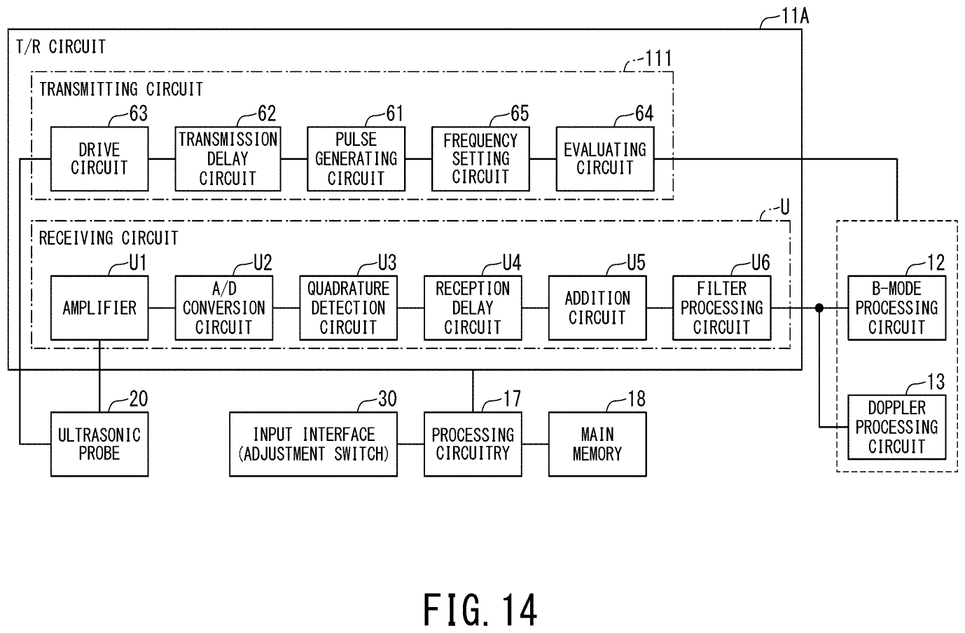

The T/R circuit 11A includes a transmitting circuit 111 and a receiving circuit U (shown in FIG. 14). The T/R circuit 11A controls the transmission directivity and the reception directivity in the transmission/reception of ultrasonic waves under the control of the processing circuitry 17. The case where the T/R circuit 11A is provided in the ultrasonic diagnostic apparatus 10A will be described, but the T/R circuit 11A may be provided in the ultrasonic probe 20, or may be provided in both the ultrasonic diagnostic apparatus 10A and the ultrasonic probe 20. The T/R circuit 11A is one example of a transmitter-and-receiver.

The transmitting circuit 111 supplies a drive signal to the ultrasonic transducer of the ultrasonic probe 20. The configuration of the transmitting circuit 111 will be described later with reference to FIG. 14. The receiving circuit U receives the received signal received by the ultrasonic transducer and performs various processing on the received signal to generate echo data. The configuration of the receiving circuit U will be described later with reference to FIG. 14.

FIG. 14 is a block diagram showing a configuration of the T/R circuit 11A. FIG. 14 shows a transmitting circuit 111 and a receiving circuit U provided in the T/R circuit 11A. The receiving circuit U includes an amplifier U1, an A/D conversion circuit U2, a quadrature detection circuit U3, a reception delay circuit U4, an adder circuit U5, and a filter processing circuit U6. The receiving circuit U receives the echo signal received by the ultrasonic vibrator and performs various processing on the echo signal to generate echo data.

The amplifier U1, the A/D conversion circuit U2, the quadrature detection circuit U3, the reception delay circuit U4, and the addition circuit U5 respectively have the same functions as the amplifier 51, the A/D conversion circuit 52, the quadrature detection circuit 53, the reception delay circuit 54, and the addition circuit 55, shown in FIG. 4. Therefore, these explanations will be omitted. The filter processing circuit U6 has a function of applying an arbitrary complex reception filter (including a real number reception filter) to the I/Q signal which is the output of the addition circuit U5, and a function of outputting the I/Q signal after the complex reception filter is applied to the B-mode processing circuit 12, the Doppler processing circuit 13, and the transmitting circuit 111.

In the above description of the receiving circuit U, a case where the receiving circuit U has a configuration for performing I/Q beamforming will be described. In I/Q beamforming, an RF signal is orthogonally detected, converted into an I/Q baseband, and then delayed and added to generate an ultrasonic image. However, it is not limited to that case. The receiving circuit U may have a configuration for performing RF beamforming. In RF beamforming, after delay addition of RF signals, quadrature detection is performed and converted into I/Q signals composed of I signal and Q signal to generate an ultrasonic image.

The transmitting circuit 111 includes a pulse generating circuit 61, a transmission delay circuit 62, a drive circuit 63, an evaluating circuit 64, and a frequency setting circuit 65. The evaluating circuit 64 may be provided in the B-mode processing circuit 12 (or the Doppler processing circuit 13) instead of the T/R circuit 11.

The pulse generating circuit 61 repeatedly generates a rate pulse for forming a transmission ultrasonic wave at a predetermined rate frequency under the control of the processing circuitry 17.

The transmission delay circuit 62 converges the ultrasonic waves generated from the ultrasonic transducer of the ultrasonic probe 20 into a beam shape, and gives a delay time of each piezoelectric transducer necessary for determining the transmission directivity to each rate pulse generated by the pulse generating circuit 61 under the control of the processing circuitry 17.

The drive circuit 63 applies a drive pulse to the ultrasonic transducer at a timing based on the rate pulse under the control of the processing circuitry 17. The drive circuit 63 is one example of a drive unit.

In the embodiment, the transmission frequency (lowest frequency/medium frequency/highest frequency) of the ultrasonic wave transmitted from the ultrasonic probe 20 can be selected by a user interface (UI). However, optimization of transmission frequency selection including sensitivity is desired. This is because there may be an operator who does not even touch the UI, or the operator may not have the skill to select an appropriate transmission frequency. Therefore, the transmitting circuit 111 provided in the T/R circuit 11A has the evaluating circuit 64 and the frequency setting circuit 65. As a result, the degree of beam penetration to the deep portion is evaluated as the sensitivity, and an appropriate transmission frequency is automatically selected according to the degree of beam penetration to the deep portion. Here, the lowest frequency is also called “PEN: Penetration”. The medium frequency is also called “GEN: general”. The highest frequency is also called “RES: Resolution”.

Under the control of the processing circuitry 17, the evaluating circuit 64 analyzes the received signal at a predetermined depth based on the received signal of the ultrasonic wave, and evaluates the degree of beam penetration to the deep portion. For example, the evaluating circuit 64 evaluates degree of beam penetration to the deep portion using the signal to noise (SN) ratio of the deep determination region described later. It is based on B-mode data (or Doppler data) as raw data from the B-mode processing circuit 12 (or Doppler processing circuit 13). The evaluating circuit 64 may analyze the received signal having a predetermined depth based on the B-mode image data (or Doppler image data) after scan conversion from the image generating circuit 14. The evaluating circuit 64 is one example of an evaluating unit.

The frequency setting circuit 65 sets the transmission frequency based on the result of the evaluating circuit 64 under the control of the processing circuitry 17. As a result, the drive circuit 63 can generate a drive pulse based on the transmission frequency set by the frequency setting circuit 65, and apply the drive pulse to the ultrasonic transducer of the ultrasonic probe 20 at a timing based on the rate pulse. The frequency setting circuit 65 is one example of the frequency setting unit.

Subsequently, an operation of the ultrasonic diagnostic apparatus 10A will be described. The ultrasonic diagnostic apparatus 10A samples at a low transmission frequency (for example, the lowest frequency (PEN) of the switchable transmission frequencies), and controls to switch to a high transmission frequency (for example, medium frequency (GEN)) when the SN ratio of the image has a margin.

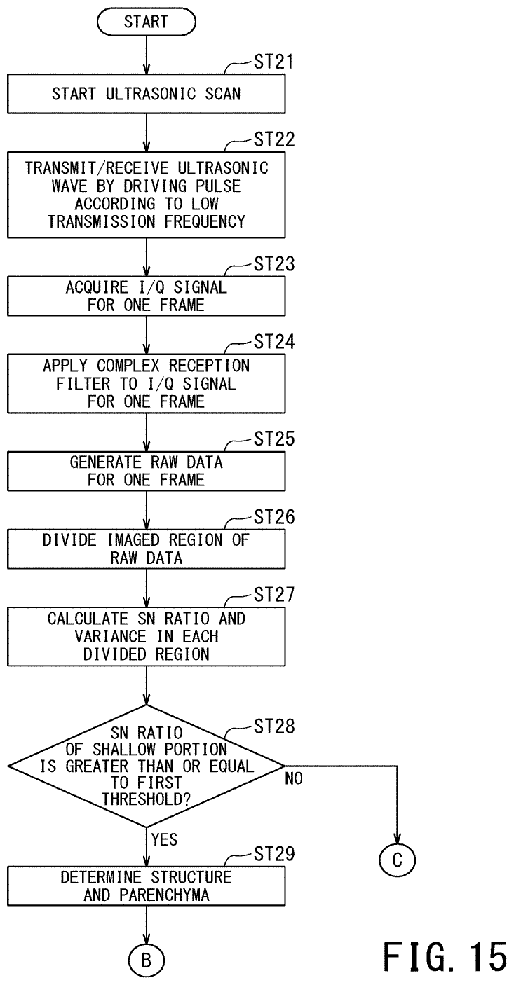

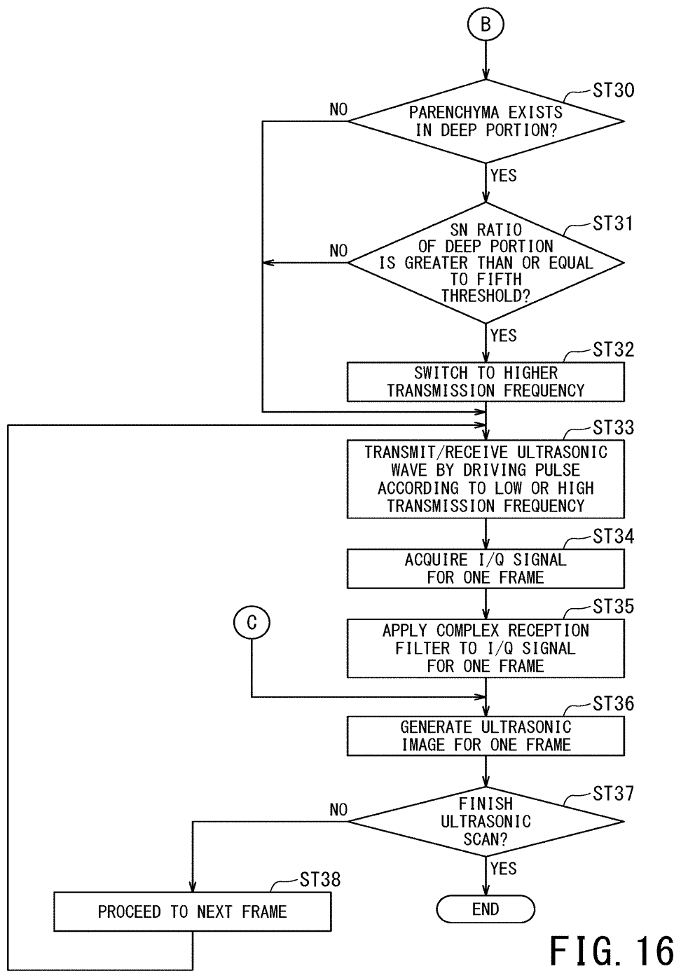

Each of FIGS. 15 and 16 is a diagram showing an operation of the ultrasonic diagnostic apparatus 10A as a flowchart. In FIGS. 15 and 16, the reference numerals “ST” with numbers indicate each step of the flowchart. Note that, in FIGS. 15 and 16, the case of I/Q beamforming, that is, the case where the reception filter is a complex reception filter will be described as an example.

As shown in FIG. 15, the processing circuitry 17 of the ultrasonic diagnostic apparatus 10A controls the T/R circuit 11A and the like to start an ultrasonic scan using the ultrasonic probe 20 (step ST21). The T/R circuit 11A controls the ultrasonic probe 20 by a drive pulse having a low transmission frequency (e.g., the lowest frequency (PEN)) set by the frequency setting circuit 65 to transmit/receive ultrasonic waves (step ST22). The filter processing circuit U6 acquires the I/Q signal for one frame which is the output of the addition circuit U5 (step ST23).

The filter processing circuit U6 applies an arbitrary complex reception filter to the I/Q signal for one frame acquired in step ST23 (step ST24). The B-mode processing circuit 12 generates B-mode data as raw data for one frame based on the I/Q signal to which an arbitrary complex reception filter is applied in step ST24 (step ST25).

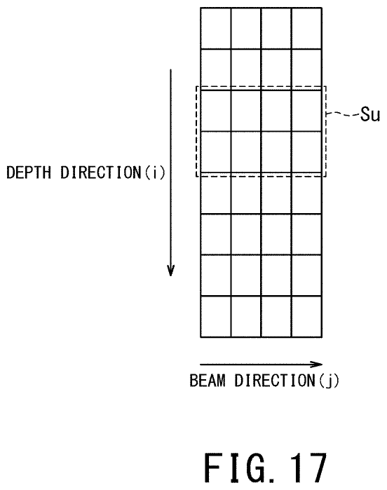

In steps ST26 to ST31, the evaluating circuit 64 analyzes the received signal at a predetermined depth to evaluate the degree of beam penetration to the deep portion. First, the evaluating circuit 64 divides the image region formed by the B-mode data for one frame generated in step ST25 into multiple divided regions such that each region has multiple pixels (step ST26). The evaluating circuit 64 calculates the SN ratio of each divided region after division and the variance of the signals in step ST26 based on the B-mode data for one frame generated in step ST25 (step ST27). FIG. 17 shows 4×8 divided regions in the image region formed by one frame of B-mode data. Each divided region contains N (N: an integer greater than or equal to 2) pixels.

In step ST27, the evaluating circuit 64 acquires the signal mean of each divided region by the following equation (1) (signal mean). It is based on the average of multiple signals corresponding to the respective multiple pixels in each divided area. In step ST27, the evaluating circuit 64 acquires the noise mean of each divided region by the following equation (2). It is based on the average of multiple noises corresponding to the respective multiple pixels in each divided region. Then, in step ST27, the evaluating circuit 64 calculates the SN ratio (SNR) [dB] of each divided region by the following equations (3) and (4). It is based on the signal average and noise average of multiple pixels in each divided area. Further, in step ST27, the evaluating circuit 64 calculates the variance (Var) of each divided region by the following equation (5). It is based on multiple signals corresponding to the respective pixels in each divided region and a signal mean of each divided region.

$$\begin{matrix}Signal mean = \frac{\sum Signals}{N} & ( 1 ) \\ Noise mean = \frac{\sum Noises}{N} & ( 2 ) \\ SNR = \frac{Signal mean}{Noise mean} & ( 3 ) \\ SNR [ dB ] = 20 {log}_{10} ( SNR ) & ( 4 ) \\ Var = \frac{\sum {( Signals - Signal mean )}^{2}}{Signal mean} & ( 5 )\end{matrix}$$

The evaluating circuit 64 determines whether or not the SN ratio of the shallow determination region in the image region formed by the B-mode data for one frame is equal to or greater than the first threshold value (step ST28). The I/Q signal that is the basis of the B-mode data includes a received signal in a scanned state or a received signal in an aerial state in which the ultrasonic probe 20 is left in the air away from the body surface of the subject. The state in which the ultrasonic probe 20 is left in the air away from the body surface of the subject is synonymous with the state in which ultrasonic waves are not transmitted to the subject. Therefore, in step ST28, the evaluating circuit 64 does not evaluate the degree of beam penetration to the deep portion when the ultrasonic probe 20 is left in the air.

FIG. 17 is a diagram showing an example of a shallow determination region set in the image region formed by B-mode data for one frame.

As shown in FIG. 17, the image region formed by the B-mode data for one frame has a total of 32 divided regions having 8 steps in the depth direction (i) and 4 rows in the beam direction j. The evaluating circuit 64 sets the determination region Su in a shallow portion of the image region. Here, among the image regions, the shallow determination region Su is set in the third and fourth stages from the top, and eight shallow divided regions corresponding to the shallow portion are set.

The evaluating circuit 64 evaluates the signal to noise ratio in the eight shallow divided regions, and determines whether the ultrasonic probe 20 is in a scanning state of being applied to the body surface of the subject or is in an aerial state where the ultrasonic probe 20 is away from the body surface of the subject. When the SN ratio of at least one shallow divided region out of the eight shallow divided regions is equal to or greater than the first threshold value, the evaluating circuit 64 may determine that the scan state is in effect. Alternatively, the evaluating circuit 64 may determine that it is in the scan state only when the SN ratios of all the eight shallow divided regions are equal to or higher than the first threshold value. Alternatively, the evaluating circuit 64 may set only one divided region as the shallow divided region, and may determine that the scan state is in the scan state when the SN ratio of the shallow divided region is equal to or greater than the first threshold value.

Returning to the description of FIG. 15, if it is determined as “NO” in step ST28, that is, if it is determined that the SN ratio of the shallow determination region in the image region is less than the first threshold value, the process proceeds to step ST36 (in FIG. 16). That is, the frequency setting circuit 65 does not change the transmission frequency when the SN ratio in the shallow determination region is less than the first threshold value. On the other hand, if it is determined as “YES” in step ST28, that is, if it is determined that the SN ratio of the shallow determination region in the image region is equal to or higher than the first threshold value, the evaluating circuit 64 determines that the scan state is in effect, and determines the structure or parenchyma of each divided region (step ST29). In the embodiment, the parenchyma refers to an organ such as a liver.

In step ST29, the evaluating circuit 64 determines whether each of the 4×8 divided regions is a structure or a parenchyma. This is because the presence of a structure having high brightness makes it difficult to evaluate the degree of beam penetration to the deep portion. If it is a case where: the SN ratio of each divided region is equal to or greater than the first threshold value; and the variance of the divided region is equal to or greater than the second threshold value, the evaluating circuit 64 determines that such a divided region corresponds to the structure. If it is a case where: the SN ratio of each divided region is equal to or greater than the first threshold; the variance of the divided region is less than the second threshold; and the variance of the divided region is equal to or greater than the third threshold (third threshold<second threshold), the evaluating circuit 64 determines that such a divided region corresponds to the parenchyma. On the other hand, if it is a case where the SN ratio of each divided region is less than the first threshold value, or where the variance of each divided region is less than the third threshold, the evaluating circuit 64 determines that such a divided region does not correspond to the structure or the parenchyma.

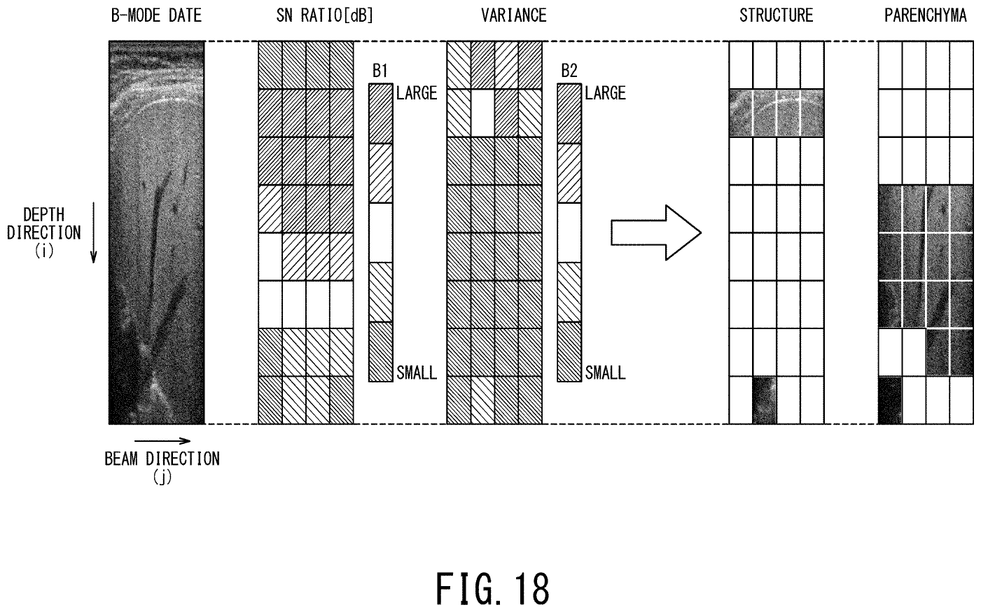

FIG. 18 is a diagram for explaining a method of determining the structure and the parenchyma based on B-mode data for one frame.

The leftmost end of FIG. 18 shows B-mode data as raw data for one frame. The second from the left shows the distribution of the SN ratio in the 4×8 divided regions. Each divided region is colored by the color arrangement bar Bl according to the magnitude of the SN ratio. The third from the left shows the distribution of the variance of the signals in each of the 4×8 divided regions. Each divided region is colored by the color arrangement bar B2 according to the magnitude of the variance of the signals.

The second from the right in FIG. 18 shows a divided region determined to be a structure in the B-mode data. These divided regions have a relatively large signal to noise ratio and a relatively large signal dispersion. The rightmost end of FIG. 18 shows a divided region determined to be substantial in the B-mode data. These divided regions have a relatively large signal to noise ratio, while the variance of the signals is relatively small. The evaluating circuit 64 may display the distribution of the SN ratio and the distribution of the variance shown in FIG. 18 on the display 40 at an arbitrary timing.

Proceeding to the description of FIG. 16, the evaluating circuit 64 determines whether or not the parenchyma determined by step ST29 exists in the deep determination region of the image region formed by the B-mode data for one frame (Step ST30). If it is determined as “YES” in step ST30, that is, if it is determined that the parenchyma exists in the deep determination region of the image region formed by one frame of B-mode data, the evaluating circuit 64 determines the SN of the deep determination region. It is determined whether or not the ratio is equal to or greater than the fifth threshold value (step ST31). In step ST27 (shown in FIG. 15), the evaluating circuit 64 calculated the SN ratio and the signal variance for all of the divided regions, but the present invention is not limited to that case. For example, steps ST28 to ST30 may be omitted. In this case, in step ST27, the evaluating circuit 64 may calculate only the SN ratio for only the divided region belonging to the deep determination region among the divided regions for step ST31.

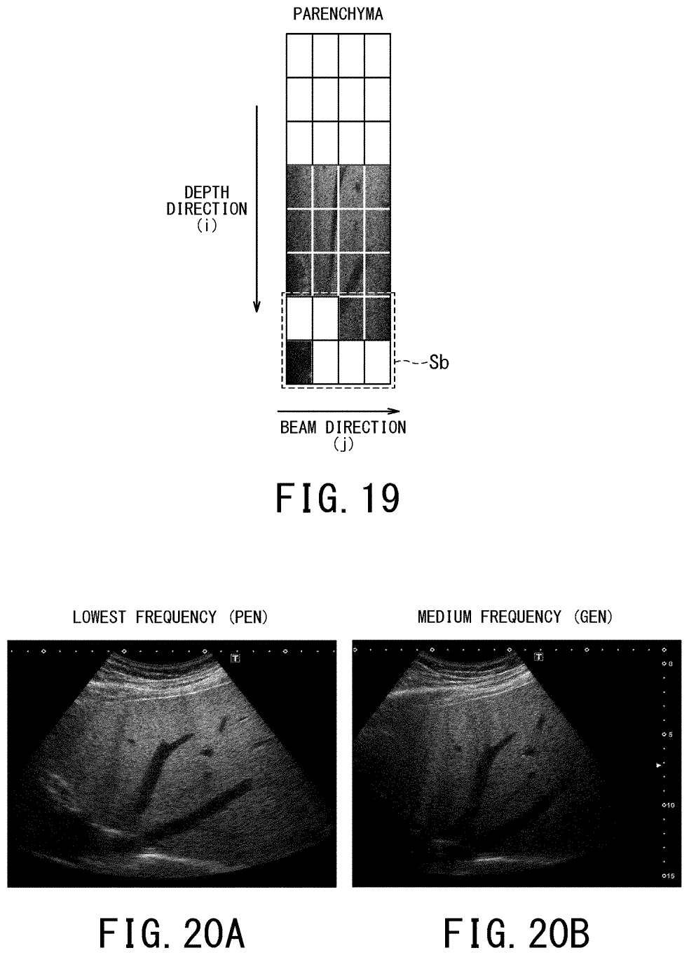

FIG. 19 is a diagram showing an example of a deep determination region set in the image region formed by B-mode data for one frame. FIG. 19 is a diagram showing steps ST30 and ST31.

As shown in FIG. 19, the evaluating circuit 64 sets a deep determination region Sb in the deep portion of the image region formed by the B-mode data for one frame. Here, in the image region, the deep determination region Sb is set in the lowermost two stages, and eight deep divided regions corresponding to the deep portion are set. In step ST30, the evaluating circuit 64 determines that three of the eight deep divided regions belonging to the determination region Sb belong to the parenchyma.

Then, in step ST31, the evaluating circuit 64 evaluates the SN ratio in the three deep divided regions belonging to the parenchyma among the eight deep divided regions. The evaluating circuit 64 may determine whether or not the average of the three SN ratios corresponding to the three deep divided regions belonging to the parenchyma is equal to or greater than the fifth threshold value. The evaluating circuit 64 may determine whether or not the average of the eight SN ratios corresponding to all eight deep divided regions is equal to or greater than the fifth threshold value.

Returning to the explanation of FIG. 16, if it is determined as “YES” in step ST31, that is, if it is determined that the SN ratio of the deep determination region in the B-mode data region for one frame is equal to or higher than the fifth threshold value, the frequency setting circuit 65 determines that the degree of beam penetration to the deep portion is high for the B-mode data for the one frame. Then, the frequency setting circuit 65 sets the transmission frequency higher than that set in step ST22, and switches to a higher transmission frequency (step ST32). This is because when the degree of beam penetration to the deep portion is high, even if the transmission frequency is changed to a high value, the effect on the deep imaging is considered to be small.

If it is determined as “NO” in step ST30, or if it is determined as “NO” in step ST31, the T/R circuit 11A does not change the transmission frequency set by the frequency setting circuit 65. The T/R circuit 11A controls the ultrasonic probe 20 by a drive pulse having a low transmission frequency similar to step ST22 (shown in FIG. 15) to transmit/receive ultrasonic waves (step ST33). On the other hand, the T/R circuit 11A controls the ultrasonic probe 20 by the drive pulse of the high transmission frequency after switching in step ST32 to transmit/receive ultrasonic waves (step ST33). The filter processing circuit U6 acquires the I/Q signal for one frame which is the output of the addition circuit U5 (step ST34).

The filter processing circuit U6 applies an arbitrary complex reception filter to the I/Q signal for one frame acquired in step ST34 (step ST35). The B-mode processing circuit 12 (or Doppler processing circuit 13) and the image generating circuit 14 generate an ultrasonic image based on the I/Q signal for one frame to which the complex reception filter is applied in step ST35 (step ST36). An ultrasonic image for one frame is generated based on the I/Q signal for one frame to which the complex reception filter is applied acquired in step ST24 or ST35 (step ST36).

FIG. 20 is a diagram showing an ultrasonic image when the transmission frequency is controlled. FIG. 20A shows a B-mode image in the case of a low transmission frequency, for example, the lowest frequency (PEN). FIG. 20B shows a B-mode image for high transmission frequencies, for example, the medium frequency (GEN). The imaging target (site) of the B-mode image shown in FIG. 20 is the liver.

In the B-mode image shown in FIG. 20A based on the transmission and reception of ultrasonic waves having the lowest frequency, even the form of the deep portion can be sufficiently visually recognized. However, if the transmission frequency is arbitrarily switched from the lowest frequency to the medium frequency or the highest frequency, as shown in FIG. 20B, the degree of beam penetration to the deep portion becomes low, and it becomes difficult to visually recognize the deep portion. Therefore, the ultrasonic diagnostic apparatus 10A evaluates the degree of beam penetration to the deep portion from the B-mode image based on the ultrasonic transmission/reception of the lowest frequency, thereby switching the transmission frequency from the lowest frequency to the medium frequency or from the medium frequency to the highest frequency.

Returning to the description of FIG. 16, the processing circuitry 17 determines whether or not to finish the ultrasonic scan started by step ST21 (shown in FIG. 15) (step ST37). For example, the processing circuitry 17 determines whether or not to finish the ultrasonic scan by the finish operation by the operator via the input interface 30. If it is determined as “NO” in step ST37, that is, if it is determined that the ultrasonic scan started in step ST21 is not finished, the process proceeds to the next frame (step ST38), and the T/R circuit 11A controls the ultrasonic probe 20 by a drive pulse having a low or high transmission frequency after being switched to transmit/receive ultrasonic waves (step ST33).

On the other hand, If it is determined as “YES” in step ST37, that is, if it is determined that the ultrasonic scan started in step ST21 is finished, the processing circuitry 17 of the ultrasonic diagnostic apparatus 10A controls the T/R circuit 11A and the like to finish the ultrasonic scan using the ultrasonic probe 20. The processing circuitry 17 can display the ultrasonic image (e.g., B-mode image) generated in step ST36 on the display 40. Further, the processing circuitry 17 can also display the ultrasonic images before and after the switching of the transmission frequency on the display 40 in parallel. Further, the processing circuitry 17 can also display a message on the display 40 with respect to the ultrasonic image after the transmission frequency is switched. The message is for the operator to select the transmission frequency after switching as it is.

Therefore, when the transmission frequency set in step ST22 is the lowest frequency (PEN), the transmission frequency after switching in step ST32 is the medium frequency (GEN) or the highest frequency (RES). When the transmission frequency set in step ST22 is the lowest frequency (PEN), and when the transmission frequency after switching in step ST32 is the highest frequency (RES), it is determined whether or not the SN ratio of the deep determination region is equal to or greater than the sixth threshold value (sixth threshold value>fifth threshold value). Alternatively, when the transmission frequency set in step ST22 is the medium frequency, the transmission frequency after switching in step ST32 is the highest frequency.

Further, the transmission frequency may be switched stepwise. For example, the transmission frequency is set to the lowest frequency (step ST22). When the SN ratio in the deep determination region is equal to or greater than the fifth threshold value, the transmission frequency is switched from the lowest frequency to the higher medium frequency (step ST32). Subsequently, the transmission frequency is set to the medium frequency after switching (step ST22), and it is determined whether the SN ratio of the deep determination region is equal to or higher than the fifth threshold value. Then, when the SN ratio in the deep determination region is equal to or higher than the fifth threshold value, the transmission frequency is switched from the medium frequency to the high maximum frequency (step ST32).

In the above description, it has been described that the evaluation by step ST26 is started immediately after the start of the ultrasonic scan by step ST21, but the present invention is not limited to this case. For example, the evaluation starting from step ST26 may be triggered in: change in the position or angle of the ultrasonic probe 20 during ultrasonic scanning; change of information of ultrasonic images (scan conditions such as display depth); and scan-hold operation. Further, the evaluation target is not limited to the raw data acquired live, and may be the raw data of the past image.

Further, the ultrasonic images corresponding to the transmission frequencies are generated according to the flowcharts shown in FIGS. 15 and 16, thereby the processing circuitry 17 may arrange the ultrasonic images in descending order of the deep SN ratio and display them on the display 40. In this case, the operator selects a predetermined ultrasonic image, and the frequency setting circuit 65 sets the transmission frequency at which the ultrasonic image is acquired.

As described above, according to the ultrasonic diagnostic apparatus 10A, it is possible to suppress deterioration of image quality due to ultrasonic attenuation by controlling the transmission frequency according to the degree of beam penetration to the deep portion. Thereby, it is possible to provide a high-quality ultrasonic image.

6. Modification

In the ultrasonic diagnostic apparatus 10A, the transmission frequency is switched from the low transmission frequency to the high transmission frequency when the SN ratio of the deep determination region at the low transmission frequency is equal to or higher than the second threshold value. However, it is not limited to that case. For example, when the SN ratio of the deep determination region at a high transmission frequency is less than the fifth threshold value, the ultrasonic diagnostic apparatus 10A may determine that the degree of beam penetration to the deep portion is low, and may switch the transmission frequency from a high transmission frequency to a low transmission frequency. However, if simple and highly reproducible transmission frequency control is desired without considering the difference between each test of the subject, it is preferable to control the transmission frequency from a low transmission frequency to a high transmission frequency.

7. Ultrasonic Diagnostic Apparatus According to Third Embodiment

The above-mentioned ultrasonic diagnostic apparatus realizes the provision of high-quality ultrasonic images by controlling the transmission frequency of ultrasonic waves or controlling a complex reception filter. However, it may be possible to provide a high-quality ultrasonic image by both controlling the transmission frequency of the ultrasonic wave and controlling the complex reception filter. That is, the above-mentioned first and second embodiments may be combined. A case will be described below as an ultrasonic diagnostic apparatus according to a third embodiment.

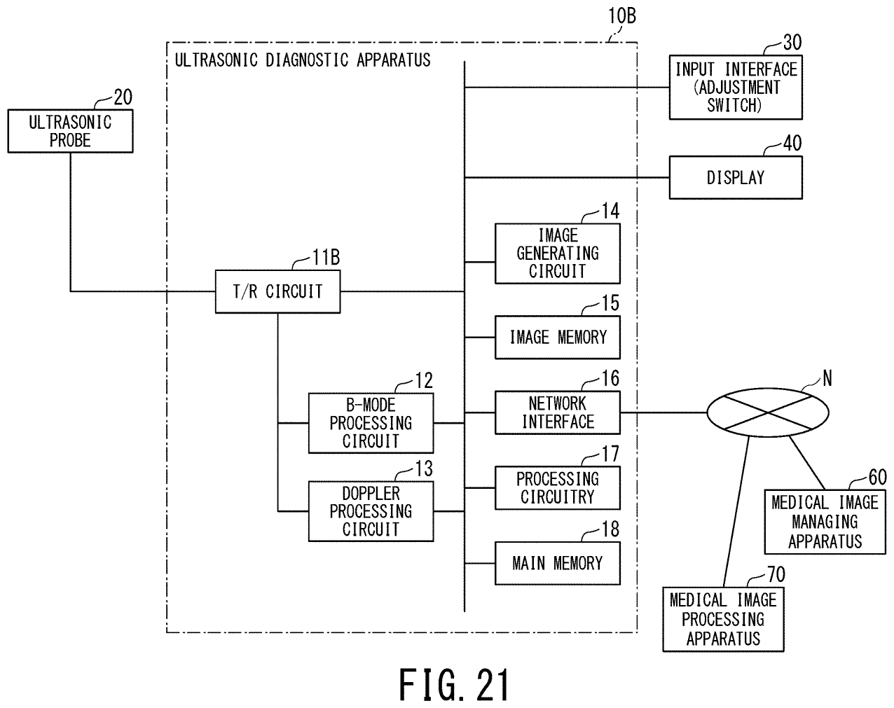

FIG. 21 is a schematic diagram showing a configuration of an ultrasonic diagnostic apparatus according to a third embodiment.

FIG. 21 shows the ultrasonic diagnostic apparatus 10B according to the third embodiment. Further, FIG. 21 shows an ultrasonic probe 20, an input interface 30, and a display 40. A device in which at least one of the ultrasonic probe 20, the input interface 30, and the display 40 is added to the ultrasonic diagnostic apparatus 10 B may be referred to as “ultrasonic diagnostic apparatus”. In the following description, a case where the ultrasonic probe 20, the input interface 30, and the display 40 that are all provided outside the ultrasonic diagnostic apparatus 10B will be described.

The ultrasonic diagnostic apparatus 10B includes a T/R circuit 11B, a B-mode processing circuit 12, a Doppler processing circuit 13, an image generation circuit 14, an image memory 15, a network interface 16, a processing circuitry 17, and a main memory 18. The circuits 11B, 12 to 14 are configured by an integrated circuit for a specific application or the like. However, the present invention is not limited to this case, and all or a part of the functions of the circuits 11B, 12 to 14 may be realized by the processing circuitry 17 executing the program.

In FIG. 21, the same parts as those shown in FIG. 1 are designated by the same reference numerals, and the description thereof will be omitted.

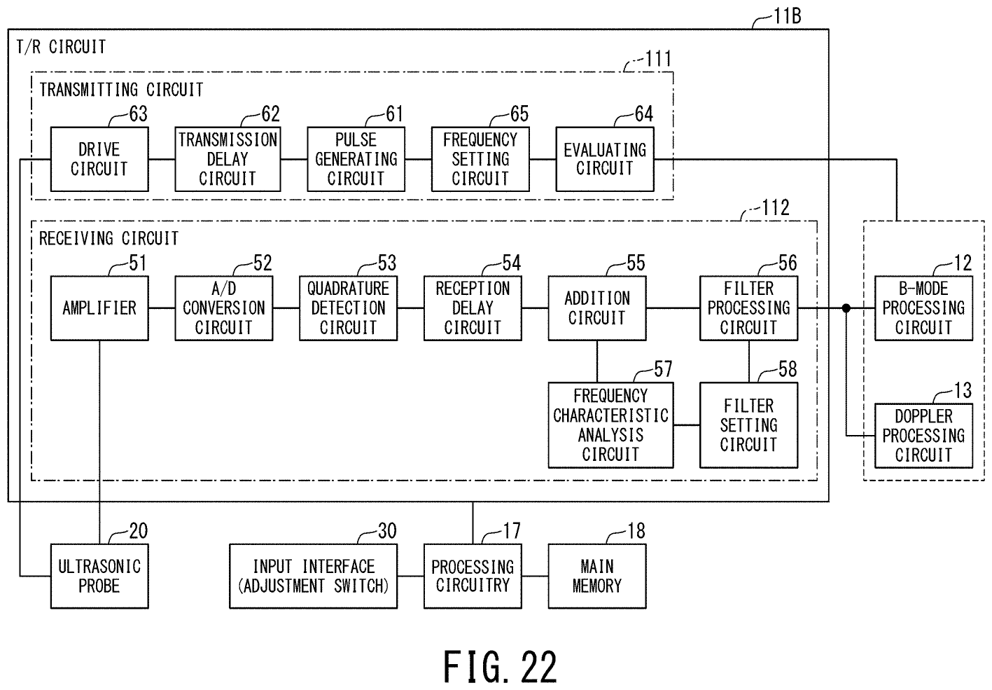

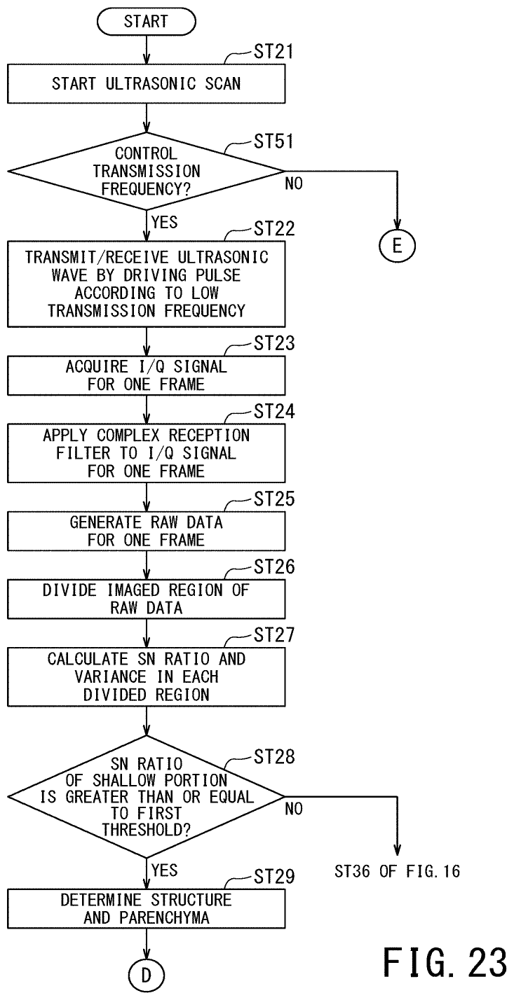

The T/R circuit 11B includes a transmitting circuit 111 and a receiving circuit 112 (shown in FIG. 22). The T/R circuit 11B controls the transmission directivity and the reception directivity in the transmission/reception of ultrasonic waves under the control of the processing circuit 17. A case where the T/R circuit 11B is provided in the ultrasonic diagnostic apparatus 10B will be described. However, the T/R circuit 11B may be provided in the ultrasonic probe 20, or may be provided in both the ultrasonic diagnostic apparatus 10B and the ultrasonic probe 20. The T/R circuit 11 B is one example of a transmitter-and-receiver.

FIG. 22 is a block diagram showing a configuration of the T/R circuit 11B.

FIG. 22 shows a transmitting circuit 111 and a receiving circuit 112 provided in the T/R circuit 11B. The transmitting circuit 111 includes a pulse generating circuit 61, a transmission delay circuit 62, and a drive circuit 63, an evaluating circuit 64 and frequency setting circuit 65, and supplies a drive signal to the ultrasonic transducer of the ultrasonic probe 20. The receiving circuit 112 includes an amplifier 51, an A/D conversion circuit 52, a quadrature detection circuit 53, a reception delay circuit 54, an addition circuit 55, a filter processing circuit 56, a frequency characteristic analysis circuit 57 and a filter setting circuit 58, and receives the echo signal received by the ultrasonic transducer and performs various processing on the echo signal to generate echo data. All the functions of circuits 61 to 65 and 51 to 58 are realized by one processing circuit executing a program, or some of them may be realized by executing programs by different processing circuits.

In the transmitting circuit 111 of FIG. 22, the same members as those of the transmitting circuit 111 shown in FIG. 14 are designated by the same reference numerals, and the description thereof will be omitted. Further, in the receiving circuit 112 of FIG. 22, the same members as those of the receiving circuit 112 shown in FIG. 4 are designated by the same reference numerals, and the description thereof will be omitted.

Subsequently, the operation of the ultrasonic diagnostic apparatus 10 B will be described. It should be noted that it is possible to select with a preset whether to control only the ultrasonic transmission frequency, or only the complex reception filter, or both.

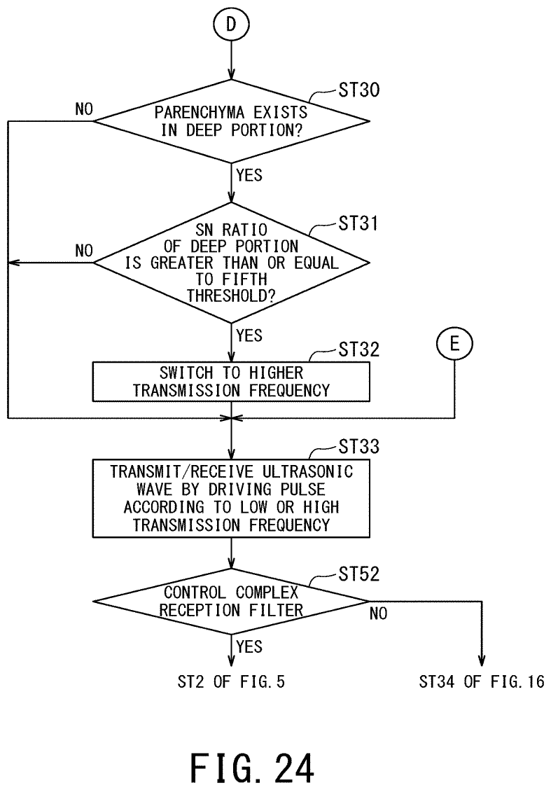

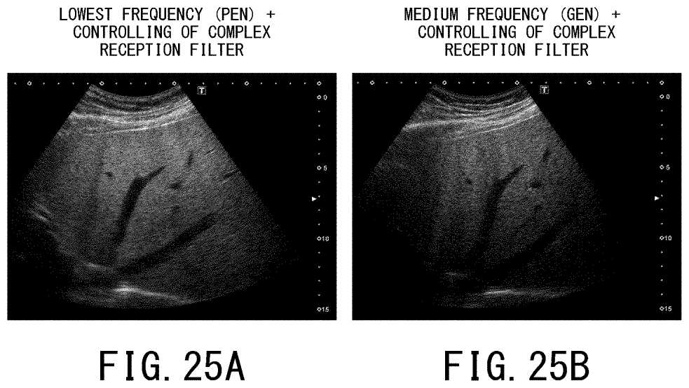

Each of FIGS. 23 and 24 is a diagram showing an operation of the ultrasonic diagnostic apparatus 10 B as a flowchart. In FIGS. 23 and 24, the reference numerals “ST” with numbers indicate each step of the flowchart. In addition, in FIG. 23 and FIG. 24, the case of I/Q beamforming, that is, the case where the reception filter is a complex reception filter will be described as an example. Further, in FIGS. 23 and 24, the same steps as the steps in the flowcharts of FIGS. 15 and 16 are designated by the same reference numerals, and the description thereof will be omitted.

As shown in FIG. 23, after the ultrasonic scan is started (step ST21), the processing circuitry 17 of the ultrasonic diagnostic apparatus 10 B determines whether or not to control the transmission frequency (step ST51). For example, the processing circuitry 17 determines whether or not to control the transmission frequency by the finish operation by the operator via the input interface 30. If it is determined as “YES” in step ST51, that is, if it is determined that the transmission frequency is controlled, the T/R circuit 11B controls the ultrasonic probe 20 by the drive pulse of the low transmission frequency set by the frequency setting circuit 65, and transmits/receives ultrasonic waves (step ST22).

On the other hand, if it is determined as “NO” in step ST51, that is, the transmission frequency is not controlled, the process proceeds to step ST33 shown in FIG. 24.

Proceeding to the description of FIG. 24, ultrasonic waves are transmitted and received by a drive pulse corresponding to a low or high transmission frequency after switching, then the processing circuitry 17 of the ultrasonic diagnostic apparatus 10 B determines whether or not to control the complex reception filter (step ST52). For example, the processing circuitry 17 determines whether or not to control the complex reception filter by the finish operation by the operator via the input interface 30.

If it is determined as “YES” in step ST52, that is, if it is determined that the complex reception filter is controlled, the process proceeds to step ST2 in FIG. 5. On the other hand, if it is determined as “NO” in step ST52, that is, if it is determined that the complex reception filter is not controlled, the process proceeds to step ST34

in FIG. 16. With ultrasonic diagnostic apparatus 10B, any one of: the case where only controlling the complex reception filter (first embodiment); the case where only controlling the transmission frequency (second embodiment); and the case where controlling both the complex reception filter and controlling the transmission frequency can be selected arbitrarily. If it is determined as “NO” in step ST51 and as “YES” in step ST52, the ultrasonic diagnostic apparatus 10 B can only control the complex reception filter. If it is determined as “YES” in step ST51 and as “NO” in step ST52, the ultrasonic diagnostic apparatus 10B can only control the transmission frequency. If it is determined as “YES” in step ST51 and as “YES” in step ST52, the ultrasonic diagnostic apparatus 10B can control both the complex reception filter and the transmission frequency.

Each of FIGS. 25A and 25B is a diagram showing an ultrasonic image when the transmission frequency and the complex reception filter are controlled. FIG. 25A shows a B-mode image in the case of a low transmission frequency, for example, the lowest frequency (PEN), and in the case of controlling the complex reception filter according to the depth. FIG. 25B shows a B-mode image in the case of a high transmission frequency, for example, a medium frequency (GEN), and in the case where the complex reception filter is controlled according to the depth. The imaging target (site) of the B-mode image shown in FIG. 25 is the liver.

In FIG. 25A, the image quality is optimized as compared with FIG. 20A. Further, in the B-mode image shown in FIG. 25A based on the ultrasonic transmission/reception of the lowest frequency and to which the complex reception filter according to the depth is applied, even the form of the deep portion can be sufficiently visually recognized. On the other hand, if the transmission frequency is arbitrarily switched from the lowest frequency to the medium frequency or the highest frequency, as shown in FIG. 25B, the degree of beam penetration to the deep portion becomes low, and it becomes difficult to visually recognize the deep portion. Therefore, the ultrasonic diagnostic apparatus 10B evaluates the degree of beam penetration to the deep portion from the B-mode image based on the ultrasonic transmission/reception of the lowest frequency even when the complex reception filter according to the depth is applied. As a result, the ultrasonic diagnostic apparatus 10B switches the transmission frequency from the lowest frequency to the medium frequency or from the medium frequency to the highest frequency.

When both the transmission frequency control and the complex reception filter control are performed, multiple transmission frequencies can be scanned. In that case, the degree of beam penetration to the deep portion is evaluated at each transmission frequency, and the transmission frequency when the deep SN ratio is the highest is adopted. This is to optimize the image quality condition in the desired reception band.

As described above, according to the ultrasonic diagnostic apparatus 10B, the transmission frequency is controlled according to the degree of beam penetration to the deep portion, and the complex reception filter is controlled according to the depth. As a result, it is possible to suppress image quality deterioration due to ultrasonic attenuation. Thereby, it is possible to provide a high-quality ultrasonic image. Further, in the ultrasonic diagnostic apparatus 10B, both the control of the ultrasonic transmission frequency (second embodiment) and the control of the complex reception filter (first embodiment) can be combined. Even when the transmission frequency is lowered to reduce the image quality when only the ultrasonic transmission frequency is controlled, it is possible to compensate the disadvantage by controlling the complex reception filter.

According to at least one embodiment described above, it is possible to suppress deterioration of image quality due to ultrasonic attenuation, so it is possible to provide a high-quality ultrasonic image.

While certain embodiments have been described, these embodiments have been presented by way of example only, and are not intended to limit the scope of the inventions. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an evaluating circuit configured to analyze a received signal of a predetermined depth based on received signals of ultrasonic wave to evaluate a degree of beam penetration to a deep portion;
a frequency setting circuit configured to set a transmission frequency based on a result evaluated by the evaluating circuit; and
a drive circuit configured to generate a drive pulse based on the set transmission frequency.

2. The ultrasonic diagnostic apparatus according to claim 1, wherein
the evaluating circuit is configured to evaluate the degree of beam penetration to the deep portion based on a signal to noise (SN) ratio of a deep portion.

3. The ultrasonic diagnostic apparatus according to claim 1, wherein
the received signals include a received signal in a scanned state or a received signal in a state in which ultrasonic waves are not transmitted to a subject.

4. The ultrasonic diagnostic apparatus according to claim 1, wherein
the received signals are received signals corresponding to a lowest switchable transmission frequency.

5. The ultrasonic diagnostic apparatus according to claim 4, wherein
the frequency setting circuit is configured to set a frequency higher than a minimum frequency as the transmission frequency to be set based on the result evaluated by the evaluating circuit.

6. The ultrasonic diagnostic apparatus according to claim 1, wherein
the evaluating circuit is configured to evaluate the degree of beam penetration to the deep portion based on raw data before scan conversion as the received signals.

7. The ultrasonic diagnostic apparatus according to claim 6, wherein
the evaluating circuit is configured to
divide an image region formed by the raw data into divided regions,
determine whether or not each of the divided regions belongs to a parenchyma based on the SN ratio of each of the divided regions and variance of signals of each of the divided regions, and
compare, when a deep divided region corresponding to a deep portion belongs to the parenchyma among the divided regions, the SN ratio of the deep divided region with a threshold value, and
the frequency setting circuit is configured to
set, when the SN ratio of the deep divided region is equal to or greater than the threshold value, a high transmission frequency corresponding to the received signals.

8. The ultrasonic diagnostic apparatus according to claim 6, wherein
the evaluating circuit is configured to
divide an image region formed by the raw data into divided regions, and
compare the SN ratio of a shallow divided region corresponding to a shallow portion among the divided regions with a threshold value, and
the frequency setting circuit is configured to
not change, when the SN ratio of the shallow divided region is less than the threshold value, the transmission frequency corresponding to the received signals.

9. The ultrasonic diagnostic apparatus according to claim 1, further configured to:
a frequency characteristic analysis circuit configured to perform frequency analysis of the received signal of the predetermined depth to acquire frequency characteristic based on the received signals; and
a filter setting circuit configured to set a reception filter that corrects the frequency characteristic of the predetermined depth such that the frequency characteristic of the predetermined depth exhibits a predetermined frequency characteristic; and
a frequency processing circuit configured to apply the reception filter by feeding back the reception signal of the predetermined depth.

10. The ultrasonic diagnostic apparatus according to claim 9, wherein
the frequency characteristic analysis circuit is configured to perform frequency analysis of the received signal in each region of interest according to the depth to acquire frequency characteristic for each region of interest,
the filter setting circuit is configured to set a reception filter that corrects the frequency characteristic of each region of interest for each region of interest such that the frequency characteristic of each region of interest show the predetermined frequency characteristic, and
the filter processing circuit is configured to apply the reception filter by feeding back the reception filter for each region of interest to the reception signal in each region of interest.

11. The ultrasonic diagnostic apparatus according to claim 10, wherein
the filter setting circuit is configured to, when an ultrasonic image is generated by compounding frequency components, set the reception filter for each frequency component set in each of the regions of interest.

12. The ultrasonic diagnostic apparatus according to claim 10, wherein
the frequency characteristic analysis circuit is configured to, when the received signal in each region of interest is frequency-analyzed, set each region of interest to include a central position in a scanning direction in the image region.

13. The ultrasonic diagnostic apparatus according to claim 12, wherein
the frequency characteristic analysis circuit is configured to set regions of interest at a same depth, and frequency-analyzes a region of interest selected from the set regions of interest.

14. The ultrasonic diagnostic apparatus according to claim 13, wherein
the frequency characteristic analysis circuit is configured to frequency-analyze the region of interest whose SN is higher than a threshold value among the regions of interest.

15. The ultrasonic diagnostic apparatus according to claim 9, wherein
the received signal is an I/Q (In-phase/Quadrature-phase) signal, and the filter coefficient of the received filter is a complex coefficient.

16. The ultrasonic diagnostic apparatus according to claim 9, wherein
the filter setting circuit is configured to set the reception filter such that the frequency characteristic of the received signal are substantially flat over a wide band.

17. The ultrasonic diagnostic apparatus according to claim 16, wherein
the filter setting circuit is configured to set the reception filter such that the frequency characteristic of the received signal are symmetrical on a low frequency side and a high frequency side with a center frequency as a center.

18. The ultrasonic diagnostic apparatus according to claim 9, further comprising:
an adjustment switch configured to adjust the frequency characteristic of the reception filter.

19. The ultrasonic diagnostic apparatus according to claim 9, wherein
when it is determined that change in a value indicating scan cross-section is equal to or greater than a threshold value, and when it is determined after the determination that change in a value indicating the scan cross-section is less than a threshold value, the frequency characteristic analysis circuit is configured to perform frequency analysis of the received signals again.

20. The ultrasonic diagnostic apparatus according to claim 19, wherein
the value indicating the scan cross-section is a value indicating a position or an angle of the ultrasonic probe corresponding to the scan cross-section.

21. The ultrasonic diagnostic apparatus according to claim 19, wherein
the value indicating the scan cross-section is a luminance value of the ultrasonic image corresponding to the scan cross-section.

22. The ultrasonic diagnostic apparatus according to claim 9, wherein
the filter processing circuit is configured to acquire and use, when it is determined in a series of ultrasonic examinations that a scan cross-section at a same position as in a past has been scanned, a reception filter corresponding to the scan cross-section.
